SEQUENCE LISTING (64 sequences)

SEQ ID NO: 1 - protein, 10 aa, Artificial sequence
  Synthetic peptide
RRRDDDSDDD

SEQ ID NO: 2 - protein, 23 aa, Artificial sequence
  Synthetic peptide
KKKKERLLDDRHDSGLDSMKDEE

SEQ ID NO: 3 - protein, 21 aa, Artificial sequence
  Synthetic peptide
RRELVEPLTPSGEAPNQALLR

SEQ ID NO: 4 - protein, 16 aa, Artificial sequence
  Synthetic peptide
ERMRPRKRQGSVRRRV

SEQ ID NO: 5 - protein, 9 aa, Artificial sequence
  Synthetic peptide
RRRLSSLRA

SEQ ID NO: 6 - protein, 12 aa, Artificial sequence
  Synthetic peptide
EAIYAAPFAKKK

SEQ ID NO: 7 - protein, 7 aa, Artificial sequence
  Synthetic peptide
RPRAATF

SEQ ID NO: 8 - protein, 22 aa, Artificial sequence
  Synthetic peptide
RRRPASVPPSPSLSRHSSHQRR

SEQ ID NO: 9 - protein, 14 aa, Artificial sequence
  Synthetic peptide
KKKSPGEYVNIEFG

SEQ ID NO: 10 - protein, 9 aa, Artificial sequence
  Synthetic peptide
APRTPGGRR

SEQ ID NO: 11 - protein, 9 aa, Artificial sequence
  Synthetic peptide
ISNRRGTRG

SEQ ID NO: 12 - protein, 11 aa, Artificial sequence
  Synthetic peptide
QKRPSQRSKYL

SEQ ID NO: 13 - protein, 9 aa, Artificial sequence
  Synthetic peptide
KKRNRTLTK

SEQ ID NO: 14 - protein, 15 aa, Artificial sequence
  Synthetic peptide
KVEKIGEGTYGVVYK

SEQ ID NO: 15 - protein, 14 aa, Artificial sequence
  Synthetic peptide
ARKRERTYSFGHHA

SEQ ID NO: 16 - protein, 10 aa, Artificial sequence
  Synthetic peptide
SSVSLTRSLP

SEQ ID NO: 17 - protein, 18 aa, Artificial sequence
  Synthetic peptide
RRKDLHDDEEDEAMSITA

SEQ ID NO: 18 - protein, 10 aa, Artificial sequence
  Synthetic peptide
RRADDSDDDD

SEQ ID NO: 19 - protein, 10 aa, Artificial sequence
  Synthetic peptide
HATPPKKKRK

SEQ ID NO: 20 - protein, 10 aa, Artificial sequence
  Synthetic peptide
PKTPKKAKKL

SEQ ID NO: 21 - protein, 10 aa, Artificial sequence
  Synthetic peptide
GRSTESRAAV

SEQ ID NO: 22 - protein, 9 aa, Artificial sequence
  Synthetic peptide
APRTPGGRR

SEQ ID NO: 23 - protein, 14 aa, Artificial sequence
  Synthetic peptide
RRGRTGRGRRGIFR

SEQ ID NO: 24 - protein, 8 aa, Artificial sequence
  Synthetic peptide
QLRSQRSL

SEQ ID NO: 25 - protein, 10 aa, Artificial sequence
  Synthetic peptide
RREDAEAARG

SEQ ID NO: 26 - protein, 7 aa, Artificial sequence
  Synthetic peptide
LRRWSLG

SEQ ID NO: 27 - protein, 21 aa, Artificial sequence
  Synthetic peptide
KRELVEPLTPSGEAPNQALLR

SEQ ID NO: 28 - protein, 21 aa, Artificial sequence
  Synthetic peptide
ADPDHDHTGFLTEYVATRWRR

SEQ ID NO: 29 - protein, 10 aa, Artificial sequence
  Synthetic peptide
KGAEAVTSPR

SEQ ID NO: 30 - protein, 7 aa, Artificial sequence
  Synthetic peptide
AKRESAA

SEQ ID NO: 31 - protein, 10 aa, Artificial sequence
  Synthetic peptide
KKLRRTLSVA

SEQ ID NO: 32 - protein, 15 aa, Artificial sequence
  Synthetic peptide
HMRSAMSGLHLVKRR

SEQ ID NO: 33 - protein, 11 aa, Artificial sequence
  Synthetic peptide
RRRDDDXSDDD

SEQ ID NO: 34 - protein, 24 aa, Artificial sequence
  Synthetic peptide
KKKKERLLDDRHDSGLDXSMKDEE

SEQ ID NO: 35 - protein, 22 aa, Artificial sequence
  Synthetic peptide
RRELVEPLTPXSGEAPNQALLR

SEQ ID NO: 36 - protein, 17 aa, Artificial sequence
  Synthetic peptide
ERMRPRKRQGXSVRRRV

SEQ ID NO: 37 - protein, 10 aa, Artificial sequence
  Synthetic peptide
RRRLXSSLRA

SEQ ID NO: 38 - protein, 13 aa, Artificial sequence
  Synthetic peptide
EAIXYAAPFAKKK

SEQ ID NO: 39 - protein, 8 aa, Artificial sequence
  Synthetic peptide
RPRAAXTF

SEQ ID NO: 40 - protein, 23 aa, Artificial sequence
  Synthetic peptide
RRRPAXSVPPSPSLSRHSSHQRR

SEQ ID NO: 41 - protein, 15 aa, Artificial sequence
  Synthetic peptide
KKKSPGEXYVNIEFG

SEQ ID NO: 42 - protein, 10 aa, Artificial sequence
  Synthetic peptide
APRXTPGGRR

SEQ ID NO: 43 - protein, 10 aa, Artificial sequence
  Synthetic peptide
ISNRRGXTRG

SEQ ID NO: 44 - protein, 12 aa, Artificial sequence
  Synthetic peptide
QKRPXSQRSKYL

SEQ ID NO: 45 - protein, 10 aa, Artificial sequence
  Synthetic peptide
KKRNRXTLTK

SEQ ID NO: 46 - protein, 16 aa, Artificial sequence
  Synthetic peptide
KVEKIGEGTXTGVVYK

SEQ ID NO: 47 - protein, 15 aa, Artificial sequence
  Synthetic peptide
ARKRERXTYSFGHHA

SEQ ID NO: 48 - protein, 11 aa, Artificial sequence
  Synthetic peptide
SSVXSLTRSLP

SEQ ID NO: 49 - protein, 19 aa, Artificial sequence
  Synthetic peptide
RRKDLHDDEEDEAMXSITA

SEQ ID NO: 50 - protein, 11 aa, Artificial sequence
  Synthetic peptide
RRADDXSDDDD

SEQ ID NO: 51 - protein, 11 aa, Artificial sequence
  Synthetic peptide
HAXTPPKKKRK

SEQ ID NO: 52 - protein, 11 aa, Artificial sequence
  Synthetic peptide
PKXTPKKAKKL

SEQ ID NO: 53 - protein, 14 aa, Artificial sequence
  Synthetic peptide
GPHRXSTPESRAAV

SEQ ID NO: 54 - protein, 10 aa, Artificial sequence
  Synthetic peptide
APRXTPGGRR

SEQ ID NO: 55 - protein, 15 aa, Artificial sequence
  Synthetic peptide
RRGRXTGRGRRGIFR

SEQ ID NO: 56 - protein, 12 aa, Artificial sequence
  Synthetic peptide
QKRPXSQRSKYL

SEQ ID NO: 57 - protein, 14 aa, Artificial sequence
  Synthetic peptide
RRLIEDAEXYAARG

SEQ ID NO: 58 - protein, 8 aa, Artificial sequence
  Synthetic peptide
LRRWXSLG

SEQ ID NO: 59 - protein, 22 aa, Artificial sequence
  Synthetic peptide
KRELVEPLTPXSGEAPNQALLR

SEQ ID NO: 60 - protein, 22 aa, Artificial sequence
  Synthetic peptide
ADPDHDHXTGFLTEYVATRWRR

SEQ ID NO: 61 - protein, 11 aa, Artificial sequence
  Synthetic peptide
KGAEAVXTSPR

SEQ ID NO: 62 - protein, 8 aa, Artificial sequence
  Synthetic peptide
AKREXSAA

SEQ ID NO: 63 - protein, 11 aa, Artificial sequence
  Synthetic peptide
KKLRRTLXSVA

SEQ ID NO: 64 - protein, 16 aa, Artificial sequence
  Synthetic peptide
KMXRSAMSGLHLVKRR

(19) United States
(12) Patent Application Publication
(10) Pub. No.: US 2009/0215098 A1
(43) Pub. Date: Aug. 27, 2009

(54) QUANTIFICATION OF ENZYME ACTIVITY BY MASS SPECTROMETRY

(75) Inventors: Pedro Rodriguez Cutillas, Surrey (GB); Bart Vanhaesebroeck, London (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignees: UCL BUSINESS PLC., LONDON (GB); LUDWIG INSTITUTE FOR CANCER RESEARCH, NEW YORK, NY (US)

(21) Appl. No.: 12/298,810

(22) PCT Filed: Apr. 25, 2007
(86) PCT No.: PCT/US07/67358

§ 371 Date: Apr. 9, 2009

Related U.S. Application Data
(60) Provisional application No. 60/796,168, filed on Apr. 28, 2006.

(51) Int. Cl. C12Q 1/48; C12Q 1/00

(52) U.S. Cl. 435/15; 435/4

(57) ABSTRACT

The disclosure relates to methods of quantitatively analyzing the enzymatic activity of enzymes in samples containing a plurality of enzymes, using mass spectrometry. Isotopically labeled standards are employed. Purified enzymes and enzymes from crude cell lysates may be analyzed using the disclosed methods. As little as 0.02 pg of cell lysate may be detected. Also disclosed are kits for providing compositions so as to practice the disclosed methods.

### CROSS-REFERENCE TO RELATED APPLICATIONS

This application claims the benefit of priority of U.S. Provisional Application No. 60/796,168, filed Apr. 28, 2006, the disclosures of which is expressly incorporated by reference in its entirety.

### BACKGROUND OF THE DISCLOSURE

1. Field of the Invention

The present invention relates to materials and methods for quantification of enzymes or enzyme activity in a sample. In particular, the present invention relates to methods of quantifying enzyme activity using spectroscopy such as mass spectroscopy. The information obtained is valuable for biological research; pharmaceutical research and development; medical diagnosis, prophylaxis, and therapy; forensics; and many other practical applications.

2. Related Technology

Because many enzymes act aberrantly in a variety of disease states, including cancer, it is valuable to have a means of quantifying enzymatic activity of samples. Quantitative measurements of specific enzymatic activity may lead to rapid diagnosis of patients' disease states and may also lead to swift evaluation of targeted therapies for specific disease states. The means for accomplishing this quantitative analysis has not been proposed in a manner that would allow for rapid and systematic analysis of samples.

The detection and effective therapeutic blockade of signal transduction pathways in cancer is seriously hampered by the lack of simple tools to quantify changes in pathway activation status. Techniques currently available involve purification, or semi-purification, of samples or enzymes of interest from other enzymes (see, e.g., Cutillas et al, Mol Cell Proteomics 4(8):1038-51 (2005), Gerber et al., Proc Natl Acad Sci USA 100(12): 6940-45 (2003), Ballif et al., Proc Natl Acad Sci USA 102(3): 667-72 (2005), Loog, et al., J Biomolecular Screening 10(4): 320-8 (2005), Beausoleil et al., Proc Natl Acad Sci USA 101(33): 12130-5 (2004), Rush et al., Nature Biotechnol 23(1): 94-101 (2005), Sonoda et al., Bioorg Med Chem Lett 14:847-50 (2004), Kratchmarova et al., Science 308:1472-7 (2005), Luo et al., Endocrinology 146(10):4410-6 (2005), Smolka et al., Mol Cell Proteomics 1(1):19-29 (2002), Goshe et al., Curr Opin Biotechnol 14(1):101-9 (2003), and Ducret et al., Protein Sci 7:706-19 (1998)). Often these other methods cannot give absolute quantification of enzyme activity, only relative quantification; and these other methods require large amounts of cells for meaningful measurements.

Purification of the enzymes of interest prior to analysis of their activity can hamper the rapid assessment of a sample. Complexities in sample preparation or in analysis slow down a clinician's ability to assess a patient's diagnosis cost-effectively, rapidly, and accurately. The current means for using mass spectrometry for enzyme activity do not allow for rapid or multi-faceted analysis of enzymes.

### SUMMARY OF THE INVENTION

The present disclosure addresses the need for materials and methods for analyzing enzyme activities of samples to yield quantitative data that may be compared across samples.

One aspect of the invention is a quantitative method for detecting the activity of an enzyme in a sample that contains a plurality of enzymes. For example, in one variation, the method comprises: incubating the sample with a substrate composition that comprises a first substrate which is specific for a first enzyme that is known or suspected of being in the sample, wherein the first enzyme is a kinase and wherein the incubating is under conditions effective to permit a first reaction between the first enzyme and the first substrate to produce a first product; combining an aliquot from the first reaction with a measured quantity of a first standard of a known molecular weight to form a first mixture for analysis; and analyzing the first mixture by mass spectrometry to determine the quantity of the first product that is present in the first mixture, wherein the quantity of the first product provides a quantitative measurement of the activity of the first enzyme in the sample. Although many embodiments of the enzyme are described in the context of kinases, the invention can be used to assay other classes of enzymes, too.

In another variation, the method comprises: incubating the sample with a substrate composition to start an enzymatic reaction, wherein the substrate composition comprises a first substrate that is specific for a first enzyme that is known or suspected of being in the sample, and wherein the incubating is under conditions effective to permit a first reaction between the first enzyme and the first substrate to produce a first product; combining an aliquot from the enzymatic reaction with a measured quantity of a first standard of known molecular weight to form a first mixture for analysis; and analyzing the first mixture by liquid chromatography-mass spectrometry (LC-MS) to determine the quantity of the first product that is present in the first mixture, wherein the quantity of the first product provides a quantitative measurement of the activity of the first enzyme in the sample.

The term “enzyme” refers to any protein that has a biological activity of modifying, or catalyzing the modification of, a molecule referred to as a “substrate” into another molecule or molecules referred to as a “product.” For example, a kinase is an enzyme that modifies a substrate molecule by adding a phosphate moiety, to create a phosphorylated product molecule. Kinases can be protein kinases, lipid kinases, carbohydrate kinases such as phosphofructokinase, or small molecule kinases such as pyruvate kinase. Specific protein kinases which may be used in the disclosed methods are listed below in Table 1. An enzyme may include one or more polypeptide chains as well as modifications (e.g., glycosylation, phosphorylation, methylation, etc.) or co-factors (e.g., metal ions).

The term “an enzyme” in the preceding description of the method refers to one or more enzymes. As described in greater detail below, the method can be practiced in a multiplex fashion to analyze the activity of multiple enzymes at once. Each enzyme modifies (e.g., catalyzes the modification of) a substrate to form a product. The use of ordinals (e.g., “first” or “second” or “third” and so forth) to refer to elements such as an enzyme, a substrate, a standard, or a product is for clarity purposes only, to identify which enzyme, substrate, product, and standard are related to each other and to distinguish the substrate, standard, and product of one enzyme from the substrate, product, and standard of another enzyme that is assayed. The ordinals are not meant to imply any particular relationship or required order between the multiple enzymes that are to be assayed.

In some cases, the enzyme participates in a cellular signaling pathway. Cellular signaling pathways are the biochemical mechanisms by which cells convert extracellular signals into the required cellular response. Cellular signaling pathways are generally discussed in Hunter, “Signaling—2000 and Beyond,” Cell 100:113-117 (2000), the entirety of which is incorporated by reference herein. These signaling pathways involve a multitude of different enzymes and the methods disclosed herein can provide a measurement of the signaling pathway as a whole, not just of specific enzymes within the pathway. Some examples of signaling pathways, the activity of which can be measured using the methods disclosed herein, include P13K/AKT pathways; Ras/Raf/MEK/Erk pathways; MAP kinase pathways; JAK/STAT pathways; mTOR/TSC pathways; heterotrimeric G protein pathways; PKA pathways; PLC/PKC pathways; NK-kappaB pathways; cell cycle pathways (cell cycle kinases); TGF-beta pathways; TLR pathways; Notch pathways; Wnt pathways; Nutrient signaling pathways (AMPK signaling); cell-cell and cell:substratum adhesion pathways (such as cadherin or integrins); stress signaling pathways (e.g., high/low salt, heat, radiation); cytokine signaling pathways; antigen receptor signaling pathways; and co-stimulatory immune signaling pathways. In some cases when the enzyme is involved in a cellular signaling pathway, the enzyme is an intracellular enzyme, i.e., an enzyme found only within a cell.

As applied to this method, the term “quantitative” refers to the method's ability to provide an absolute measurement of enzymatic activity that can be compared to measurements taken at a different time or place. Quantitative measurements are more valuable for many purposes than relative measurements that can only be compared to other measurements taken at the same time that may yield information such as a ratio. As described below in greater detail, the use of a measured quantity of the standard permits quantitative calculation of the activity of an enzyme in a sample.

The term “enzyme composition” reflects the fact that the method can be practiced with impure samples that contain a plurality (two or more) of enzymes as well as other materials. For example, any biological sample or extract that contains biologically active enzymes can be used as an enzyme composition to practice methods of the invention. As described below in greater detail, whole cells or tissue samples, cell lysates, bodily fluids or secretions or excretions, plant extracts, are examples of enzyme compositions. In these contexts, plurality may refer to, tens, hundreds, thousands, or more enzymes.

The incubating step involves placing the enzyme composition and the substrate composition together under conditions wherein the enzyme is biologically active, to permit the enzyme to modify the substrate. For an enzyme composition that comprises one or more whole cells, the incubating may involve adding the substrate to the culture media of the cell, for example. For an enzyme composition that is a cell lysate, the incubating may involve mixing the enzyme and the substrate together. Factors required for enzymatic activity, such as a particular temperature or pH, salt concentration, co-factors, ATP, GTP, and the like, will generally be known for enzymes, and even when unknown, would be expected to be similar to the physiological microenvironment where the enzyme is active in vivo.

In some variations, the enzyme composition is a mixture of purified enzymes. The enzyme composition can also be all or a fraction of a cell lysate which contains enzymes from the cell. In certain cases, the lysate comes from a human or animal subject. The lysate may be of fewer than 100 cells, or fewer than 25 cells, or even fewer than 10 cells. In certain cases, the first enzyme is a kinase and, in specific embodiments, is a protein kinase or lipid kinase. In some cases, the first enzyme is an oxidoreductase, transferase, hydrolase, lyase, isomerase, or ligase.

In one embodiment, the analysis occurs by tandem mass spectrometry, which involves a first mass spectrometry analysis to isolate a fraction of the ionized sample that contains the first product and the first standard; fragmenting the first product and the first standard in the fraction; and performing a second mass spectrometry analysis after the fragmenting to quantitatively measure at least one fragment from the first product and the first standard, wherein the fragment measurements indicate the quantities of the first product and the first standard. The analysis may also be performed by conventional mass spectrometry, in which matrix assisted laser desorption ionization (MALDI) or electrospray ionization is coupled with single mass analyzers such as time of flight (TOF), quadrupoles, sectors, or ion traps. In some variations, the measurement is performed by quantitative evaluation of the unfragmented molecular ions. In a typical variation, the quantity of the first product of the enzymatic reaction is calculated by comparing mass spectrometric measurements of the first product and the first standard in the first mixture.

In some cases, the methods further include purifying the first product and first standard before the determining step to provide a purified sample for analysis. Any techniques that are useful for chemical or biochemical separation may be used for the purifying step, including the use of chromatographic techniques, affinity purification materials and methods, electrophoresis techniques, and the like. In certain cases, the purification is done by high pressure liquid chromatography (HPLC).

In some cases, the enzyme composition further includes protease inhibitors added prior to or contemporaneous to starting the enzymatic reaction. Protease inhibitors serve to inhibit degradation of the enzyme or degradation of protein substrates, products, and standards. More generally, in some variations of the invention, the method includes the addition of factors that are necessary for the enzymatic reaction, or that improve the enzymatic reaction, or that prevent degradation of the product.

In one embodiment, the first enzyme is a protein kinase such as Akt/PKB or a phosphoinosotide kinase. Kinase activity may require the availability of a phosphate donor. Thus, in some cases, the methods include addition of adenosine triphosphate (ATP) to the enzymatic reaction. In some cases, phosphatase inhibitors are included prior to or contemporaneous to starting the enzymatic reaction, to prevent degradation (dephosphorylation) of the reaction product.

In one embodiment, the substrate comprises a peptide. The peptide may be any size that is recognized and modified by the target enzyme to be assayed. Smaller peptides are preferred due to ease of manufacture and manipulation and because they may present fewer sites for modification by non-target enzymes, i.e., they may have greater enzyme specificity. In some cases, the peptide has 5 to 45 amino acid residues. A number of specific peptide sequences that are useful as substrates for certain specific enzymes are set forth below in greater detail. In certain cases, the peptide is a peptide having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31. or SEQ ID NO: 32. Numerous enzyme-substrate combinations have been described in the literature and the invention is not limited to this set of examples.

In some cases, the standard is identical to the product of the enzymatic reaction, with the proviso that the molecular weight or mass of the standard is different from the product due to an isotope incorporated into either the product or the standard. Stable isotopes (those that are not radioactive or not decaying over time) are preferred. In certain cases, the isotope is one or more of a ¹³C, ¹⁵N, and ²H.

In some variations, both the substrate and the standard further comprise a tag (e.g., polyhistidine or other peptide or epitope tag, or biotin or streptavidin tag, etc.) for use in an optional purification step. In some embodiments, the substrate includes modifications to the amino acid sequence, whereas in other embodiments, it consists essentially of amino acids only.

In certain cases, the sample is cell lysate from a human or animal subject and the human or animal subject is suspected of having a disease characterized by changes in the activity of an enzyme involved in a cellular process. In one embodiment, the disease suspected is cancer.

In some cases, the methods disclosed herein may be used to quantify the enzymatic activity of second enzyme, wherein the incubating step further comprises simultaneously incubating the enzyme composition with a second substrate that is specific for a second enzyme that differs from the first enzyme, wherein the second enzyme modifies the second substrate to form a second product; and wherein the determining step further comprises determining the quantity of the second product produced during the incubating step. In certain cases, an aliquot from the reaction is mixed with a measured quantity of a second standard of a known molecular weight to form a sample for analysis. In some cases, the first and second standards are mixed with the same aliquot to permit simultaneous mass spectrometric analysis of the first and second products. In certain cases, the method comprises determining the quantity of the second product produced during the incubating step by analyzing the sample by mass spectrometry to measure quantities of the second product and the second standard in the sample, wherein the quantity of the second product provides a quantitative measurement of the activity of the second enzyme. In the same fashion, the method can be performed to assay a third enzyme, a fourth enzyme, a fifth enzyme, and so on.

In some variations, all of the enzymes to be assayed fall within the same class (e.g., protein kinases), whereas in other variations, enzymes of different classes are assayed together.

Another aspect of the invention is a method for screening compounds in order to identify a drug candidate comprising: measuring the activity of at least one enzyme from a biological sample, using a method described herein; and comparing the activity of the at least one enzyme in the presence and absence of the at least one test compound, wherein the method identifies an inhibitor or agonist drug candidate from reduced or increased activity, respectively, of the at least one enzyme in the presence of the at leaset one test compound. In certain cases, the method comprises measuring the activity of two or more enzymes in the presence or absence of a test compound. In various embodiments, the two or more enzymes are in the same signaling pathway, such as, for example, a pathway involved in cell growth, replication, differentiation, survival, or proliferation. Identification of a test compound as an inhibitor or an agonist of a particular enzyme or group of enzymes (as in the case of two or more enzymes being studied) can be accomplished by measuring the activity of a first enzyme or signaling pathway in the absence and presence of the test compound and comparing the activities as measured in order to assess the effect the test compound has. In certain cases, the methods can be used to assess the biological activity of the compound on non-target enzymes or pathways that may be relevant to drug metabolism/clearance, drug toxicity, and side-effects. This assessment may be useful for evaluating a compound as a potential drug candidate and/or its suitability for or efficacy in clinical trials. In some cases, the method comprises additional steps to further evaluate the compound. For example, the test compound is mixed with a pharmaceutically acceptable carrier to form a composition and the composition is administered to a subject to determine the effect of the composition in vivo. The subject can be a healthy subject for safety testing and/or a diseased subject and/or a model for a disease, for purpose of therapy or proving therapeutic efficacy. In one specific embodiment, the subject is a mammalian subject.

Another aspect of the invention is a method for screening an organism for a disease, disorder, or abnormality characterized by aberrant enzymatic activity comprising: quantitatively measuring the activity of an enzyme from a biological sample from an organism (e.g., a cell lysate from at least one cell of the organism) as described herein, and comparing the measurement to a reference measurement of the activity of the enzyme, wherein the presence or absence of the abnormality is identified from the comparison. Numerous enzyme-disease associations have been described in the literature and some are summarized below. Enzymes involved in cell growth, replication, differentiation, survival, or proliferation are only the preferred enzymes for such screening. In one exemplary embodiment, the abnormality is cancer; the first enzyme is Akt/PKB or a phosphoinositide kinase; and/or the first substrate is a first peptide which is SEQ ID NO: 7. In some cases, the cell lysate is obtained from a medical biopsy from a human and snap frozen to preserve enzymatic activity. In certain cases, the reference measurement is obtained from the same organism at a different time or from a different location in the organism. In other cases, the reference measurement is obtained from cells of the same cell type, from a different organism of the same species. In still other cases, the reference measurement is a statistical measurement calculated from measurements of samples of cells of the same cell type, from multiple organisms of the same species.

In some cases, the methods disclosed herein further comprise quantitatively measuring activity of at least one positive control enzyme from the biological sample. A positive control provides assurance that the sample was not handled in a manner that caused unacceptable enzyme degradation or denaturization.

One continuing need in medicine, especially oncology and infectious diseases, is to be able to better characterize a disease in an individual patient to permit better selection of a medicament that is more likely to be therapeutically effective and/or have fewer side effects. Therefore, another aspect of the invention is a method of characterizing a disease, disorder, or abnormality comprising: quantitatively measuring the activity of at least one enzyme from a sample using any of the methods disclosed herein, wherein the sample comprises at least one cell known or suspected of being diseased isolated from a mammalian subject, or comprises a lysate of the at least one cell; comparing the measurement(s) to a reference measurement of the activity of the at least one enzyme; and characterizing the disease or disorder by identifying an enzyme with elevated activity in the at least one diseased cell compared to activity of the enzyme in non-diseased cells of the same type as the diseased cell. In certain cases, the disease is a neoplastic disease. In some embodiments, the method further comprises selecting a composition or compound for administration to the mammalian subject, wherein the composition or compound inhibits the activity of the enzyme with the elevated activity in the at least one diseased or neoplastic cell. In some cases, the method further comprises administering a composition or compound that inhibits the activity of the enzyme with the elevated activity in the at least one diseased or neoplastic cell. In certain cases, the method further comprises prescribing a medicament to the mammalian subject, wherein the medicament inhibits the activity of the enzyme with the elevated activity in the at least one diseased or neoplastic cell. In one specific embodiment, the mammalian subject is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

In some variations of the invention, the method is a method for screening for or diagnosing a disease state and the method includes a step of measuring enzyme activity as described herein in a biological sample from an organism, and a step of diagnosing the absence or the presence of the disease, or predisposition for the disease, by the measurement of enzyme activity. For example, a comparison of the measurement for a particular subject to measurements from other healthy subjects, or diseased subjects, of the same subject at an earlier point in time, indicates the proper conclusion about the disease state in the subject.

Another aspect of the invention is a quantitative method of detecting the activity of a signaling pathway in a sample having a plurality of biologically active enzymes comprising: incubating the sample with a substrate composition which comprises a first substrate that is specific for the signaling pathway, and wherein the incubating is under conditions effective to permit a first reaction between at least one enzyme of the signaling pathway and the first substrate to produce a first product; combining an aliquot from the reaction with a measured quantity of a first standard of known molecular weight to form a first mixture for analysis; and analyzing the first mixture by mass spectrometry to determine the quantity of the first product that is present in the first mixture, wherein the quantity of the first product provides a quantitative measurement of the activity of the signaling pathway in the sample. A substrate that is specific for a signaling pathway may be converted into a product by one or more enzymes involved in the pathway, but should be unmodified by other enzymes that may be presented in the sample but that do not participate in the pathway.

Another aspect of the invention is a kit comprising two or more items useful for practicing a method of the invention, packaged together. For example, in one variation, the kit comprises a plurality of substrate containers, wherein each substrate container contains at least one enzymatic substrate that an enzyme modifies to form a product and a plurality of standard containers, wherein each standard container contains at least one mass labeled standard of a known concentration, wherein the mass labeled standard is identical to one of the products, with the proviso that the product and the standard have different molecular weights due to isotopic labeling of the standard or the product. In some cases, the kit further comprises a container having protease inhibitors such as Na-p-tosyl-L-lysine chlormethyl ketone hydrochloride (TLCK), phenylmethylsulphonylfluoride (PMSF), leupeptin, pepstatin A, aprotinin, 4-(2-aminoethyl)benzenesulfonylfluoride hydrochloride (AEBSF), 6-aminohexanoic acid, antipain hydrochloride {[(S)-1-carboxy-2-phenylethyl]-carbamoyl-L-arginyl-L-valyl-arginal-phenylalanine}, benzamidine hydrochloride hydrate, bestatin hydrochloride, chymostatin, epoxysuccinyl-L-leucyl-amido-(4-guanidino)butane, ethylenediamine tetraacetic acid disodium salt, N-ethylmaleimide, and Kunitz trypsin inhibitor. In certain cases, the kit further includes a container of phosphatase inhibitors. Exemplary phosphatase inhibitors include, but are not limited to, sodium fluoride, sodium orthovanadate, ocadaic acid, Vphen, microcystin, b-glycerophosphate, lacineurin, cantharidic acid, cyclosporin A, delamethrin, dephostatin, endothall, fenvalerate, fostriecin, phenylarsine oxide, and resmethrin.

In certain cases, the kit comprises substrate which are peptide having 6 to 250 amino acid residues. In some cases, the substrates are peptides having 5 to 45 residues.

Another aspect of the invention is a composition comprising a mixture of two or more standards of known molecular weight and concentration, wherein each of the standards comprises a chemical structure identical to an enzyme product and a molecular weight different than the enzyme product due to incorporation of at least one isotopic label in the standards. In some cases, the standards comprise peptides having 5 to 45 amino acids residues. In certain cases, the composition further includes protease inhibitors and/or phosphatase inhibitors. In one embodiment, the composition is packaged in a kit further including at least one container having at least one of the enzyme substrates.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows (a) a schematic of the steps for determining the enzymatic activity of a protein kinase and (b) a standard plot of the correlation between ratio of enzymatic product to internal standard (a mass labeled enzymatic product) and known concentration of the enzymatic product, wherein the bottom table shows the recalculated concentrations based upon the ratios measured and the known concentration of internal standard;

FIG. 2. shows (a) at top, a plot of the enzymatic activity of Akt measured for various enzyme amounts, at bottom, a matrix assisted laser desorbtion ionization—time of flight mass spectroscopy (MALDI-TOF MS) spectrum using 0.02 pg of Akt/PKB (roughly 500 zmol) protein and (b) at top, representative chromatograms from LC-MS analyses obtained using different amount of cell lysate and measuring amount of product produced, at bottom, a plot of the quantitative data derived from the chromatograms;

FIG. 3. shows (a) MS quantification of kinase activity for B lymphoma cells treated with PI3K inhibitors WM or IC87114, (b) MS quantification of kinase activity for B lymphoma total cell lysates and Akt immunoprecipitates in the presence (right) or absence (absence) of the PI3K inhibitor WM, and (c) kinase activity quantification in B lymphoma cell lysates in absence (top graph) or presence (middle and bottom graph) of PI3K activators;

FIG. 4. shows (a) MS quantification of B16/B16 solid tumor cell kinase activity in absence (left) or presence (right) of the PI3K inhibitor, LY294002 and (b) kinase activity quantification of CD34⁺ CD38⁻ stem cells and CD34⁺ CD38⁺ bulk tumor fractions in four patients; and

FIG. 5 shows a multiplex analysis wherein 3 different enzymes—(a) and (d) PKC, (b) S6 p70 kinase, and (c) Erk—with four different substrates—(a) SEQ ID NO: 12; (b) SEQ ID NO: 5; (c) SEQ ID NO: 10; and (d) SEQ ID NO: 23—in the same sample were analyzed by mass spectrometry; where the first four columns correspond to reaction times 0, 10, 30, and 60 minutes, respectively, and the last column reflects all four time points in one graph for each enzyme/substrate analysis.

### DETAILED DESCRIPTION

The detection and effective therapeutic modulation (stimulation, up-regulation, inhibition, or blockade) of signal transduction pathways in human diseases, including, but not limited to, cancer, diabetes, allergies, inflammation, and neurodegenerative diseases, is seriously hampered by inadequate tools to quantify changes in pathway activation status. The techniques described here, in one embodiment, enable the measurement of signal transduction pathway activity in a biological sample (such as a tissue, fluid, or cell sample) with the sensitivity, specificity, and precision needed for providing clinically useful information. This analytical strategy may be applied to any protein or enzyme whose product or substrate is amenable to mass spectrometric detection. In preferred variations, at lease one selective substrate of the target enzyme is available. Enzymes and substrates/products involved in a signal transduction pathway provide clinically useful information about the pathway. Because this method is based upon a biochemical (e.g., enzymatic) reaction that amplifies the signal of the target molecule, it could be described as a proteomic analytical equivalent the polymerase chain reaction (PCR) used to amplify nucleic acid sequences.

In addition, the specificity of mass spectrometry as used in methods of the invention offers the opportunity of measuring several reaction products simultaneously in a fast “multiplex” format that can be automated for clinical implementation.

The mechanism of action of many pharmaceutical agents (as well as lead, pre-clinical, and clinical candidate compounds) is to modulate enzymatic activity, which is a major factor in controlling cellular and tissue biochemistry. By providing a rapid, sensitive, specific, and optionally multiplex means for analyzing enzyme activities involved in signal transduction, metabolism, and related biochemical processes, the materials and methods of the invention are useful for both drug research and development and drug prescription, administration, and patient monitoring. For example, in the field of drug development, the materials and methods of the invention are useful for assessing the biological activity of a compound on a target pathway, and also for assessing the biological activity of the compound on non-target pathways that may be relevant to drug metabolism/clearance, drug toxicity, drug-drug interactions, and side-effects.

In a typical drug screening, the activity of a system is independently measured in the absence and presence of a test compound. The affect of that test compound is evaluated as a comparison between the measured activity in the absence of the compound and the activity in the presence of the compound. The methods disclosed herein are a means of measuring the effect of a potential drug candidate in a biological system by providing quantitative measurements of activities of one or more enzymes of interest in a biological system.

It is well established that not all patients that have been diagnosed with a disease or condition will respond to the same medication in the same way, or at the same dose, or with the same side effects. The materials and methods of the invention have utility in this clinical setting as well, e.g., to identify the subpopulation of patients that are more likely to benefit from using a particular drug, targeting a specific pathway, selecting a dose or dosing regimen, and minimizing unnecessary side effects. In these ways, the materials and methods of the invention are useful for improving personalized disease therapy. Appropriateness of a particular drug may be predicted by analyzing a biological sample from a patient to determine the activity of the protein(s) on which the target enzyme acts.

Specific aberrant enzyme activity has been associated with many disease states. Enzyme activity which is aberrant is activity that is either higher or lower than an enzyme's usual activity in a population (or samples from a population) not affected by a particular disease state. By being able to quantitatively measure enzyme activity in a manner that allows meaningful comparisons between sample sets, it may be possible to identify a particular disease state, select a more effective therapy, measure efficacy of treatments for diseases, and compare different treatments. The ability to measure enzymatic or protein activity with exquisite sensitivity also has indications for predicting the future occurrence of, or early diagnosis of, diseases at a time before other, more noticeable signs or symptoms of the disease present themselves, permitting earlier treatment, prophylaxis, and/or lifestyle management decisions to prevent or delay the onset of disease. For example, cancer, diabetes, allergic reactions, inflammation, neurodegenerative diseases, and many other disease states are known to be related to aberrant enzymatic activity.

Therefore, in some embodiments, the methods described herein are directed toward characterizing a disease, disorder, or abnormality. A particular disease state may not exhibit itself the same way in all subjects. Therefore, a measurement of the activity of the enzyme or enzymes implicated in a particular disease may yield useful information with respect to the manner in which a particular disease is manifested in a specific subject. The activity of the enzyme or enzymes of the subject is then compared to the activity of a reference measurement. In some cases, the comparison is made over time, and can be used to assess the efficacy of a particular therapy or to evaluate the progression of a particular disease. In certain specific embodiments, the comparison is used to select an appropriate composition or compound for administration to the subject which is specific for the particular aberrant activity measured using the methods disclosed herein. In subjects where the aberrant activity is measured in certain enzymes, one compound or composition will be most effective, while other subjects with different aberrant activity will be best treated by a different set of compositions or compounds. The materials and methods of the invention provide information and guidance for selection of more effective compositions or compounds.

In some embodiments, the methods described herein are directed toward quantitative analysis of enzyme activities in a sample. Samples for use in the disclosed methods may be any sample that contains an enzyme which catalyzes a reaction wherein the substrate and/or product of that reaction is/are amenable to detection by mass spectrometry (MS). Substrates and products amenable to detection by MS, as used herein, are entities that have a molecular weight within the detection range of a MS instrument. In some cases, the molecular weight of the substrate and/or product may be in the range of about 250 Da to about 5000 Da. In one embodiment, substrates and/or products may be peptides. Typically, a peptide having 5 to 45 amino acid residues has a molecular weight in the range of about 550 Da to about 5000 Da. An enzyme may be amenable to assay according to the invention even if the natural substrate of the enzyme is too large or small for detection by MS. For example, if the substrate is a protein too large for accurate measurement by MS, a peptide that is similar or identical to a fragment of the protein may be a suitable synthetic substrate for resolution via MS. Alternatively, the natural substrate can be cleaved to permit analysis of a fragment that embodies the enzymatic modification and that is amenable to measurement by MS. In a preferred variation, the substrate is a synthetic substrate having a different molecular weight than the natural substrate of the enzyme that may be present in the biological sample.

The samples may be from any organism, including humans or animals, and may be either crude or purified. In some embodiments, the sample is from a human or animal subject that is suspected of suffering from a disease characterized by changed in activity of one or more enzymes involved in a cellular process. Crude samples are samples that have not undergone significant purification prior to analysis, such as gel electrophoresis or other types of purification (e.g., liquid chromatography, size exclusion chromatography, and the like). Purified samples may be samples of individually purified enzymes or samples of mixture of enzymes purified prior to sample preparation. Samples may be cell lysates, whole cell samples, biopsy samples, and the like. In some variations, the sample is snap frozen (frozen using dry ice or liquid nitrogen) after collection and kept at a temperature below −40° C. prior to analysis. The sample may be a bodily fluid, secretion, or excretion, including, but not limited to, whole blood, serum, plasma, urine, feces, semen, mucus, saliva, tears, sweat, or gastric fluids. The samples may contain more than one enzyme, and the methods may be used to detect simultaneously the activity of more than one enzyme present in the sample. In some cases, the enzyme in the sample may be immunopurified, to produce a crude purified enzyme fraction, prior to analysis. This step can be performed for any enzyme and is especially useful in cases where the substrates for the target enzyme do not show the desired specificity, or when the aim is to determine the activity of enzyme isoforms showing the same substrate specificity.

Biological samples may be concentrated or diluted prior to analysis, depending on the concentration or activity of enzyme that is expected to be present in the sample. Because the methods described herein measure enzymatic activity by detection of products of the enzymatic reaction, small amounts of enzyme present can be detected simply by allowing the enzymatic reaction to proceed for long periods of time, to convert more substrate into product. The amplification effect of the methods disclosed herein, therefore, allow for highly sensitive means of evaluating enzyme activity. Very little sample is needed for meaningful analysis. In some cases, the sample may be a cell lysate of 100 cells or less, or 25 cells or less, or 10 cells or less, or one cell or less.

Enzymes that may be evaluated using the techniques and methods disclosed herein include any enzyme involved in a cellular process, more specifically, enzymes such as kinases, oxidoreductases, transferases, hydrolases, lyases, isomerases, and ligases. In some preferred embodiments, kinases are assayed. More specifically, both protein kinases and lipid kinases may be evaluated. Lipid kinases include phosphoinositide 3-kinase.

Specific kinases contemplated for assay according to the methods disclosed herein include those listed in Table 1. Nonlimiting examples of contemplated kinase families include the cyclic nucleotide regulated protein kinase family, the diacylglycerol-activated, phospholipid-dependent protein kinase C (PKC) family, the RAC (Akt) protein kinase family, the family of kinases that phosphorylate G protein-coupled receptors, the budding yeast AGC-related protein kinase family, the kinases that phosphorylate ribosomal protein S6 family, the budding yeast DBF2/20 family, the flowering plant PVPK1 protein kinase homolog family, the kinases regulated by Ca2+/CaM and close relatives family, the KIN1/SNF1/Nim1 family, the cyclin-dependent kinases (CDKs) and close relatives family, the ERK (MAP) kinase family, the glycogen synthase kinase 3 (GSK3) family, the casein kinase II family, the Clk family, the Src family, the Tec/Atk family, the Csk family, the Fes (Fps) family, the Syk/ZAP70 family, the Tyk2/Jak1 family, the Ack family, the Focal adhesion kinase family, the Epidermal growth factor receptor family, the Eph/Elk/Eck orphan receptor family, the Axl family, the Tie/Tck family, the Platelet-derived growth factor receptor family, the Fibroblast growth factor receptor family, the Insulin receptor family, the LTK/ALK family, the Ros/Sevenless family, the Trk/Ror family, the DDR/TKT family, the Hepatocyte growth factor family, the Nematode Kin15/16 family, the Polo family, the MEK/STE7 family, the PAK/STE20 family, the MEKK/STE11 family, the NimA family, the wee1/mik1 family, Kinases involved in transcriptional control family, the Activin/TGFb receptor family, the Flowering plant putative receptor kinases and close relatives family, the PSK/PTK “mixed lineage” leucine zipper domain family, the Casein kinase I family, and the PKN prokaryotic protein kinase family.

Resources for information about kinases include Genbank, the Swiss-Protein protein knowledge database, the protein kinase resource database on the worldwide web at http://www.kinasenet.org/pkr/Welcome.do, the worldwide web database at www.kinase.com, and numberous other paper and electronic resources.

Individual kinases contemplated for analysis in the disclosed methods include, but are not limited to, cAPKα, cAPKβ, cAPKγ, EcAPKα, DC0, DC1, DC2, ApIC, SAK, DdPK1, DdPk2, TPK1, TPK2, TPK3, PKG-I, PKG-II, DG1, DG2, PKCα, PKCβ, PKCγ, DPKC53b, DPKC53e, ApII, PKCd, PKCe, PKCet, PKCth, DPKC98, ApIII, CeTPA1, CePKC1B, PKC1, pck1+, pck2+, PKCz, PKCi, PKCm, Akt1, Akt2, SmRAC, bARK1, bARK2, RhoK, GRK5, IT11, GRK6, DmGPRK1, FmGPRK2, SCH9, YPK1, YKR2, S6K, RSKIN, RSK2N, DBF2, DBF20, PVPK1, G11A, ZmPPK, ATPK5, ATPK7, ATPK64, PsPK5, DM, Sgk, Mast205, SPK1, CaMKIIα, CaMKIIβ, CaMKIIγ, CaMKIIδ, DmCamKII, CamKI, CaMKIV, DdMKCK, DUN1, PSK-H1, CMK1, CMK2, ACMPK, MLCK-K, MLCK-M, Titwn, TWITCH, MRE4, PhKgM, PhKgT, RSK1C, RSK2C, ASK1, ASK2, CDPK, AK1, OsSPK, KIN1, KIN2, kin1+, p78, SNF1, RKIN1, AKIN10, BKIN12, WPK4, nimx1+, YKL453, YCL24, MAPKAP2, PfCPK, PfPK2, CDC2Hs, Cdk2, Cdk3, Cdk4, Cdk5, Cdk6, PCTAIRE1, PCTAIRE2, PCTAIRE3, CAK/MO15, Dm2, Dm2C, Ddcdc2, DdPRK, LmmCRK1, PfC2R, EhC²R, CfCdc2R, cdc2+, CDC28, PHO85, KIN28, FpCdc2, MsCdc2b, OsC2R, ERK1, ERK2, ERK3, Jnk1, FmERKA, CeMPK1, CaERK1, KSS1, FUS3, HOG1, SLT2, spk1+, FpERK1, NTF3, FpMPK1, FpMPK2, FpMPK3, FpMPK4, FpMPK5, FpMPK6, FpMPK7, GSK3a, GSK2b, Sgg/zw3, MCK, MDS1, ASK-a, ASK-g, CKIIa, CKIIa′, DmCKII, CeCKII, TpCKII, DdCKIIa, CKA1, CKA2, SpCka1, GpCKII, CIk, PSK-G1, Doa, KNS1, PSK-H2, YAK1, dsk1+, prp1+, GTAp58, Dcdrk, CHED, CTK1, SGV1, KKIALRE, MAK, SME1, csk1+, MHK, c-Src, c-Yes, FYN, YRK, c-Fgr, LYN, HCK, LCK, BLK, TorFYK, Dsrc64, STK, SRK1, SRK2, SRK3, SRK4, Tex, Itk/Tsk, Btk, Dsrc28, DtSpk-1, Csk, Matk, c-Fes, FER, Dfps, PTK Group V, Abl, c-Abl, ARG, Dabl, Nabl, Syk2, ZAP70, Htk16, TYK2, JAK1, JAK2, HOP, ACK, GAK, EGFR, ErbB2, ErbB3, ErbB4, DER, let-23, SER, ECK, EEK, HEK, Ehk-1, Ehk-2, SEK, ELK, Cek10, Cek9, HEK2, Buk, EPH, Azl, Ark, c-Eyk, Brt/Sky, TiE, Tek, PDGFR-α, PDGFR-β, CSF1R, c-kit, Flk2, Flt1, Flt4, Flk1, Fig, Bek, FGFR-3, FGFR-4, DFGFR, INS.r, IRR, IGF1R, DILR, LTK, ALK, c-ros, 7LESS, Trk, TrkB, TrkC, TorRTK, Ror1, Ror2, Dror, DDR, TKT, MET, c-Sea, RON, Nkin15, Nkin16, RET, KLG, Nyk/RYK, TORSO, Dtrk, Plk, SNK, polo, CDC5, MEK1, MEK2, Dsor1, PBS2, wis1+, MKK1, MKK2, byr1+, STE7, PAK, STE20, MEKK, STE11, byr2, BCK1, NPK1, Mek1, MrkA, nimA, KIN3, FUSED, wee1+, mik1+. HsWee1, HRI, PKR, GCN2, c-raf, Araf, Braf, DmRaF, CeRaf, Ctr1, TGFbRII, ActRIIA, ActRIIB, TSR-1, TskL7, ALK-3, ALK-4, ALK-5, ALK-6, C14, Daft, Daf4, DmAtr-II, DmSax, SR2, SR6, Pto, TMK1, APK1, NAK, ZMPK1, PRO25, TMK1, pelle, MLK1, PTK1, CKIa, CKIb, CKId, TCK1, YCK2, HRR25, PKN1, PKN2, IRE1, CDC7, COT, YpkA, ninaC, CDC15, chk1+, NPR1, TSL, PIM1, ran1+, TTK, ELM1, VPS15, YKL516, c-mos, Pstk1, DPYK1, DPYK2, PhyCer, and GmPK6.

TABLE 1
SwissProt Accession Numbers and abbreviated gene names of exemplary protein kinases

| | | |
|---|---|---|
| P36896, ACV1B_HUMAN | Q6H9I1, ATG1_BOTCI | BMR1A_MOUSE Q05438, |
| Q61271, ACV1B_MOUSE | Q5A649, ATG1_CANAL | BMR1B_CHICK O00238, |
| P80202, ACV1B_RAT | Q6FL58, ATG1_CANGA | BMR1B_HUMAN P36898, |
| P37023, ACVL1_HUMAN | P87248, ATG1_COLLN | BMR1B_MOUSE Q04982, |
| Q61288, ACVL1_MOUSE | Q5K8D3, ATG1_CRYNE | BRAF1_CHICK P34908, |
| P80203, ACVL1_RAT | Q6BS08, ATG1_DEBHA | BRAF1_COTJA P15056, |
| Q28041, ACVR1_BOVIN | Q5BCU8, ATG1_EMENI | BRAF1_HUMAN P28028, |
| Q04771, ACVR1_HUMAN | Q6CSX2, ATG1_KLULA | BRAF1_MOUSE O22476, |
| P37172, ACVR1_MOUSE | Q52EB3, ATG1_MAGGR | BRI1_ARATH Q8GUQ5, |
| P80201, ACVR1_RAT | Q7RX99, ATG1_NEUCR | BRI1_LYCES Q8L899, |
| Q28043, ACVR2_BOVIN | Q8TFN2, ATG1_PICAN | BRI1_LYCPE Q9ZWC8, |
| P27037, ACVR2_HUMAN | Q8TGI1, ATG1_PICPA | BRL1_ARATH Q9ZPS9, |
| P27038, ACVR2_MOUSE | Q9Y7T4, ATG1_SCHPO | BRL2_ARATH Q9LJF3, |
| P38444, ACVR2_RAT | Q6C7U0, ATG1_YARLI | BRL3_ARATH Q8TDC3, |
| Q28560, ACVR2_SHEEP | P53104, ATG1_YEAST | BRSK1_HUMAN Q8IWQ3, |
| P27039, ACVR2_XENLA | Q9M3G7, ATM_ARATH | BRSK2_HUMAN O60566, |
| P54741, AFSK_STRCO | Q13315, ATM_HUMAN | BUB1B_HUMAN Q9Z1S0, |
| P54742, AFSK_STRGR | Q62388, ATM_MOUSE | BUB1B_MOUSE O43683, |
| P38080, AKL1_YEAST | Q6PQD5, ATM_PIG | BUB1_HUMAN O08901, |
| Q01314, AKT1_BOVIN | Q13535, ATR_HUMAN | BUB1_MOUSE |
| P31749, AKT1_HUMAN | Q9JKK8, ATR_MOUSE | O94751, BUB1_SCHPO |
| P31750, AKT1_MOUSE | Q96GD4, AURKB_HUMAN | P41695, BUB1_YEAST |
| P47196, AKT1_RAT | O70126, AURKB_MOUSE | Q9GKI7, C43BP_BOVIN |
| P31751, AKT2_HUMAN | Q9N0X0, AURKB_PIG | Q9Y5P4, C43BP_HUMAN |
| Q60823, AKT2_MOUSE | O55099, AURKB_RAT | Q9EQG9, C43BP_MOUSE |
| P47197, AKT2_RAT | Q9UQB9, | P43568, CAK1_YEAST |
| Q9Y243, AKT3_HUMAN | AURKC_HUMAN O88445, | Q754N7, CBK1_ASHGO |
| Q9WUA6, AKT3_MOUSE | AURKC_MOUSE Q95126, | Q6FP74, CBK1_CANGA |
| Q63484, AKT3_RAT | AVR2B_BOVIN Q13705, | Q6BLJ9, CBK1_DEBHA |
| Q96Q40, AL2S7_HUMAN | AVR2B_HUMAN P27040, | P31034, CBK1_KLULA |
| Q16671, AMHR2_HUMAN | AVR2B_MOUSE P38445, | Q6TGC6, CBK1_PNECA |
| Q62893, AMHR2_RAT | AVR2B_RAT P27041, | Q6CFS5, CBK1_YARLI |
| P10398, ARAF_HUMAN | AVR2B_XENLA Q94F62, | P53894, CBK1_YEAST |
| P04627, ARAF_MOUSE | BAK1_ARATH Q01389, | P38973, CC2H1_TRYBB |
| O19004, ARAF_PIG | BCK1_YEAST Q9NSY1, | P54664, CC2H1_TRYCO |
| P14056, ARAF_RAT | BMP2K_HUMAN Q91Z96, | P54665, CC2H2_TRYBB |
| O59790, ARK1_SCHPO | BMP2K_MOUSE Q13873, | P54666, CC2H3_TRYBB |
| P43291, ASK1_ARATH | BMPR2_HUMAN O35607, | P21127, CD2L1_HUMAN |
| P43292, ASK2_ARATH | BMPR2_MOUSE P36894, | P24788, CD2L1_MOUSE |
| Q75CH3, ATG1_ASHGO | BMR1A_HUMAN P36895, | P46892, CD2L1_RAT |
| Q9UQ88, CD2L2_HUMAN | O55076, CDK2_CRIGR | O96017, CHK2_HUMAN |
| Q14004, CD2L5_HUMAN | Q04770, CDK2_ENTHI | Q9Z265, CHK2_MOUSE |
| Q69ZA1, CD2L5_MOUSE | P24941, CDK2_HUMAN | Q8RWC9, CIPK1_ARATH |
| Q9BWU1, CD2L6_HUMAN | P48963, CDK2_MESAU | Q6X4A2, CIPK1_ORYSA |
| Q9NYV4, CD2L7_HUMAN | P97377, CDK2_MOUSE | Q9HFW2, CLA4_ASHGO |
| P24923, CDC21_MEDSA | Q63699, CDK2_RAT | O14427, CLA4_CANAL |
| P29618, CDC21_ORYSA | P23437, CDK2_XENLA | P48562, CLA4_YEAST |
| P19026, CDC21_PEA | Q00526, CDK3_HUMAN | P49759, CLK1_HUMAN |
| P35567, CDC21_XENLA | P11802, CDK4_HUMAN | P22518, CLK1_MOUSE |
| Q05006, CDC22_MEDSA | P30285, CDK4_MOUSE | P49760, CLK2_HUMAN |
| P29619, CDC22_ORYSA | P79432, CDK4_PIG | O35491, CLK2_MOUSE |
| P28567, CDC22_PEA | P35426, CDK4_RAT | P49761, CLK3_HUMAN |
| P24033, CDC22_XENLA | Q91727, CDK4_XENLA | O35492, CLK3_MOUSE |
| P43063, CDC28_CANAL | Q02399, CDK5_BOVIN | Q63117, CLK3_RAT |
| P00546, CDC28_YEAST | P48609, CDK5_DROME | Q9HAZ1, CLK4_HUMAN |
| Q38772, CDC2A_ANTMA | Q00535, CDK5_HUMAN | O35493, CLK4_MOUSE |
| P24100, CDC2A_ARATH | P49615, CDK5_MOUSE | P38679, COT1_NEUCR |
| Q38773, CDC2B_ANTMA | Q03114, CDK5_RAT | O22932, CPK11_ARATH |
| P25859, CDC2B_ARATH | P51166, CDK5_XENLA | P92937, CPK15_ARATH |
| Q38774, CDC2C_ANTMA | Q00534, CDK6_HUMAN | Q8NK05, CPK1_CRYNE |
| P23573, CDC2C_DROME | Q64261, CDK6_MOUSE | Q9LDI3, CPK24_ARATH |
| Q38775, CDC2D_ANTMA | P51953, CDK7_CARAU | Q06309, CRK1_LEIME |
| Q01917, CDC2H_CRIFA | P54685, CDK7_DICDI | Q12126, CRK1_SCHPO |
| P34117, CDC2H_DICDI | P50613, CDK7_HUMAN | P36615, CSK1_SCHPO |
| P61075, CDC2H_PLAF7 | Q03147, CDK7_MOUSE | Q08467, CSK21_ARATH |
| Q07785, CDC2H_PLAFK | P51952, CDK7_RAT | P68399, CSK21_BOVIN |
| P54119, CDC2_AJECA | P20911, CDK7_XENLA | P21868, CSK21_CHICK |
| P48734, CDC2_BOVIN | Q9VT57, CDK8_DROME | P68400, CSK21_HUMAN |
| P34556, CDC2_CAEEL | P49336, CDK8_HUMAN | Q60737, CSK21_MOUSE |
| P51958, CDC2_CARAU | P46551, CDK9_CAEEL | P33674, CSK21_RABIT |
| P93101, CDC2_CHERU | P50750, CDK9_HUMAN | P19139, CSK21_RAT |
| P13863, CDC2_CHICK | Q99J95, CDK9_MOUSE | P15790, CSK21_YEAST |
| P34112, CDC2_DICDI | Q641Z4, CDK9_RAT | Q08466, CSK22_ARATH |
| P23572, CDC2_DROME | Q96WV9, CDK9_SCHPO | P20427, CSK22_BOVIN |
| Q00646, CDC2_EMENI | O76039, CDKL5_HUMAN | P21869, CSK22_CHICK |
| P06493, CDC2_HUMAN | P62344, CDPK1_PLAF7 | P19784, CSK22_HUMAN |
| P23111, CDC2_MAIZE | P62343, CDPK1_PLAFK | O54833, CSK22_MOUSE |
| P11440, CDC2_MOUSE | Q7RAH3, CDPK1_PLAYO | P28020, CSK22_XENLA |
| Q9DGA5, CDC2_ORYCU | Q8ICR0, CDPK2_PLAF7 | P19454, CSK22_YEAST |
| Q9DGA2, CDC2_ORYJA | O15865, CDPK2_PLAFK | O64817, CSK23_ARATH |
| Q9DGD3, CDC2_ORYLA | Q9NJU9, CDPK3_PLAF7 | P18334, CSK2A_CAEEL |
| Q9DG98, CDC2_ORYLU | Q7RAV5, CDPK3_PLAYO | Q02720, CSK2A_DICDI |
| P43290, CDC2_PETHY | P62345, CDPK4_PLABA | P08181, CSK2A_DROME |
| Q9W739, CDC2_RANDY | Q8IBS5, CDPK4_PLAF7 | P28523, CSK2A_MAIZE |
| P39951, CDC2_RAT | Q7RJG2, CDPK4_PLAYO | Q8TG13, CSK2A_NEUCR |
| P04551, CDC2_SCHPO | Q09170, CDS1_SCHPO | P40231, CSK2A_SCHPO |
| Q41639, CDC2_VIGAC | P38938, CEK1_SCHPO | O76484, CSK2A_SPOFR |
| P52389, CDC2_VIGUN | O14757, CHK1_HUMAN | P28547, CSK2A_THEPA |
| P32562, CDC5_YEAST | O35280, CHK1_MOUSE | Q05609, CTR1_ARATH |
| P06243, CDC7_YEAST | P34208, CHK1_SCHPO | O14578, CTRO_HUMAN |
| Q15131, CDK10_HUMAN | P38147, CHK1_YEAST | P49025, CTRO_MOUSE |
| P43450, CDK2_CARAU | Q9U1Y5, CHK2_CAEEL | P27450, CX32_ARATH |
| P20792, DAF1_CAEEL | P51136, GSK3H_DICDI | Q966Y3, JNK_SUBDO |
| P50488, DAF4_CAEEL | P83101, GSK3H_DROME | Q09792, KAA8_SCHPO |
| P53355, DAPK1_HUMAN | P38970, HAL5_YEAST | Q09815, KAB7_SCHPO |
| Q80YE7, DAPK1_MOUSE | P83103, HASP_DROME | P31374, KAB7_YEAST |
| Q9UIK4, DAPK2_HUMAN | Q8TF76, HASP_HUMAN | Q09831, KAD5_SCHPO |
| Q8VDF3, DAPK2_MOUSE | Q9Z0R0, HASP_MOUSE | Q6L8L1, KAIC_ACAMR |
| O43293, DAPK3_HUMAN | Q86Z02, HIPK1_HUMAN | Q8YT40, KAIC_ANASP |
| O54784, DAPK3_MOUSE | O88904, HIPK1_MOUSE | Q7VAN5, KAIC_PROMA |
| O88764, DAPK3_RAT | Q9H2X6, HIPK2_HUMAN | Q7V5W7, KAIC_PROMM |
| P32328, DBF20_YEAST | Q9WUM7, HIPK2_MESAU | Q7V0C4, KAIC_PROMP |
| P22204, DBF2_YEAST | Q9QZR5, HIPK2_MOUSE | Q79V60, KAIC_SYNEL |
| O15075, DCAK1_HUMAN | Q9H422, HIPK3_HUMAN | Q8GGL1, KAIC_SYNLI |
| Q9JLM8, DCAK1_MOUSE | Q9ERH7, HIPK3_MOUSE | Q6L8L5, KAIC_SYNP2 |
| O08875, DCAK1_RAT | O88850, HIPK3_RAT | Q79PF4, KAIC_SYNP7 |
| Q8N568, DCAK2_HUMAN | Q8T0S6, HIPPO_DROME | Q8VL13, KAIC_SYNP8 |
| Q6PGN3, DCAK2_MOUSE | Q750A9, HOG1_ASHGO | Q7U8R3, KAIC_SYNPX |
| P49762, DOA_DROME | Q92207, HOG1_CANAL | Q6L8J9, KAIC_SYNVU |
| Q9Y2A5, DUET_HUMAN | Q6FIU2, HOG1_CANGA | P74646, KAIC_SYNY3 |
| P39009, DUN1_YEAST | Q9UV50, HOG1_DEBHA | Q10078, KAND_SCHPO |
| Q9Y463, DYR1B_HUMAN | P32485, HOG1_YEAST | P06244, KAPA_YEAST |
| Q9Z188, DYR1B_MOUSE | O93982, HOG1_ZYGRO | P05131, KAPB1_BOVIN |
| Q9V3D5, DYRK2_DROME | Q08732, HRK1_YEAST | P24256, KAPB2_BOVIN |
| Q92630, DYRK2_HUMAN | P50582, HSK1_SCHPO | P40376, KAPB_SCHPO |
| Q9BQI3, E2AK1_HUMAN | P57058, HUNK_HUMAN | P06245, KAPB_YEAST |
| Q9Z2R9, E2AK1_MOUSE | O88866, HUNK_MOUSE | P00517, KAPCA_BOVIN |
| P33279, E2AK1_RABIT | Q68UT7, HUNK_PANTR | Q8MJ44, KAPCA_CANFA |
| Q63185, E2AK1_RAT | Q9UPZ9, ICK_HUMAN | P25321, KAPCA_CRIGR |
| Q9P2K8, E2AK4_HUMAN | Q9JKV2, ICK_MOUSE | P17612, KAPCA_HUMAN |
| Q9QZ05, E2AK4_MOUSE | Q62726, ICK_RAT | P05132, KAPCA_MOUSE |
| P32801, ELM1_YEAST | Q6CWQ4, ICL1_KLULA | P36887, KAPCA_PIG |
| P28869, ERK1_CANAL | Q9VEZ5, IKKB_DROME | P27791, KAPCA_RAT |
| P42525, ERK1_DICDI | O14920, IKKB_HUMAN | Q9MZD9, KAPCA_SHEEP |
| P40417, ERKA_DROME | O88351, IKKB_MOUSE | P68180, KAPCB_CRIGR |
| O75460, ERN1_HUMAN | Q9QY78, IKKB_RAT | P22694, KAPCB_HUMAN |
| Q9EQY0, ERN1_MOUSE | Q13418, ILK1_HUMAN | P68181, KAPCB_MOUSE |
| Q76MJ5, ERN2_HUMAN | P57043, ILK2_HUMAN | P05383, KAPCB_PIG |
| Q9Z2E3, ERN2_MOUSE | P57044, ILK_CAVPO | P68182, KAPCB_RAT |
| Q9LYN8, EXS_ARATH | O55222, ILK_MOUSE | P22612, KAPCG_HUMAN |
| Q9NLA1, FLR4_CAEEL | Q755C4, IPL1_ASHGO | O62846, KAPCG_MACMU |
| P16892, FUS3_YEAST | Q59S66, IPL1_CANAL | P49673, KAPC_ASCSU |
| P23647, FUSED_DROME | Q6FV07, IPL1_CANGA | P21137, KAPC_CAEEL |
| Q9P7J8, GAD8_SCHPO | Q6BVA0, IPL1_DEBHA | P34099, KAPC_DICDI |
| Q9LX30, GCN2_ARATH | Q6C3J2, IPL1_YARLI | P12370, KAPC_DROME |
| Q9HGN1, GCN2_SCHPO | P38991, IPL1_YEAST | Q8SRK8, KAPC_ENCCU |
| P15442, GCN2_YEAST | P51617, IRAK1_HUMAN | P05986, KAPC_YEAST |
| Q12263, GIN4_YEAST | Q62406, IRAK1_MOUSE | P21901, KAPL_APLCA |
| O61661, GRP_DROME | Q9NWZ3, IRAK4_HUMAN | P38070, KBN8_YEAST |
| P49840, GSK3A_HUMAN | Q8R4K2, IRAK4_MOUSE | Q9UU87, KC61_SCHPO |
| P18265, GSK3A_RAT | P32361, IRE1_YEAST | P25389, KCC4_YEAST |
| P49841, GSK3B_HUMAN | Q9U6D2, JNK1_ANCCA | Q10364, KDBE_SCHPO |
| Q9WV60, GSK3B_MOUSE | Q8WQG9, JNK1_CAEEL | P16911, KDC1_DROME |
| P18266, GSK3B_RAT | P92208, JNK_DROME | O14019, KDPG_SCHPO |
| P53233, KG1Z_YEAST | P04409, KPCA_BOVIN | Q15418, KS6A1_HUMAN |
| P00516, KGP1A_BOVIN | P17252, KPCA_HUMAN | P18653, KS6A1_MOUSE |
| Q13976, KGP1A_HUMAN | P20444, KPCA_MOUSE | Q63531, KS6A1_RAT |
| O77676, KGP1A_RABIT | P10102, KPCA_RABIT | Q15349, KS6A2_HUMAN |
| P21136, KGP1B_BOVIN | P05696, KPCA_RAT | Q9WUT3, KS6A2_MOUSE |
| P14619, KGP1B_HUMAN | P05126, KPCB_BOVIN | P51812, KS6A3_HUMAN |
| Q9Z0Z0, KGP1B_MOUSE | P05771, KPCB_HUMAN | P18654, KS6A3_MOUSE |
| Q03042, KGP1_DROME | P68404, KPCB_MOUSE | O75676, KS6A4_HUMAN |
| Q03043, KGP24_DROME | P05772, KPCB_RABIT | Q9Z2B9, KS6A4_MOUSE |
| P32023, KGP25_DROME | P68403, KPCB_RAT | O75582, KS6A5_HUMAN |
| Q13237, KGP2_HUMAN | P05128, KPCG_BOVIN | Q8C050, KS6A5_MOUSE |
| Q61410, KGP2_MOUSE | P05129, KPCG_HUMAN | Q9UK32, KS6A6_HUMAN |
| Q64595, KGP2_RAT | P63318, KPCG_MOUSE | P18652, KS6AA_CHICK |
| P43637, KGS9_YEAST | P10829, KPCG_RABIT | P10665, KS6AA_XENLA |
| P38692, KIC1_YEAST; | P63319, KPCG_RAT | P10666, KS6AB_XENLA |
| P40494, KIJ5_YEAST | Q90XF2, KPCI_BRARE | Q21734, KS6A_CAEEL |
| Q38997, KIN10_ARATH | P41743, KPCI_HUMAN | P23443, KS6B1_HUMAN |
| P92958, KIN11_ARATH | Q62074, KPCI_MOUSE | Q8BSK8, KS6B1_MOUSE |
| P06242, KIN28_YEAST | Q5R4K9, KPCI_PONPY | P67998, KS6B1_RABIT |
| P13186, KIN2_YEAST | Q05513, KPCZ_HUMAN | P67999, KS6B1_RAT |
| P22209, KIN3_YEAST | Q02956, KPCZ_MOUSE | Q9UBS0, KS6B2_HUMAN |
| Q01919, KIN4_YEAST | O19111, KPCZ_RABIT | Q9Z1M4, KS6B2_MOUSE |
| P25341, KIN82_YEAST | P09217, KPCZ_RAT | Q12701, KSG1_SCHPO |
| P00513, KIPA_BPT7 | Q05652, KPEL_DROME | P38691, KSP1_YEAST |
| O74526, KJ45_SCHPO | Q39030, KPK19_ARATH | P14681, KSS1_YEAST |
| P47042, KJF7_YEAST; | P42818, KPK1_ARATH | O95835, LATS1_HUMAN |
| Q9HFF4, KK31_SCHPO | Q02595, KPK2_PLAFK | Q8BYR2, LATS1_MOUSE |
| Q9P6P3, KKB3_SCHPO | Q05999, KPK7_ARATH | Q9NRM7, LATS2_HUMAN |
| Q8N5S9, KKCC1_HUMAN | P17801, KPRO_MAIZE | Q7TSJ6, LATS2_MOUSE |
| Q8VBY2, KKCC1_MOUSE | P11801, KPSH1_HUMAN | P53667, LIMK1_HUMAN |
| P97756, KKCC1_RAT | Q08097, KR1_BHV1S | P53668, LIMK1_MOUSE |
| Q96RR4, KKCC2_HUMAN | Q04543, KR1_CHV9D | P53669, LIMK1_RAT |
| Q8C078, KKCC2_MOUSE | P28926, KR1_EHV1B | Q10156, LKH1_SCHPO |
| O88831, KKCC2_RAT | P32516, KR1_EHV1K | O61267, LOK_DROME |
| Q9UTH3, KKE1_SCHPO | P84390, KR1_EHV1V | Q02779, M3K10_HUMAN |
| Q00532, KKIA_HUMAN | P04413, KR1_HHV11 | Q16584, M3K11_HUMAN |
| P34244, KKK1_YEAST | P13287, KR1_HHV2H | Q12852, M3K12_HUMAN |
| P28708, KKL6_YEAST | P17613, KR1_PRVKA | Q60700, M3K12_MOUSE |
| P36005, KKQ1_YEAST | P24381, KR1_PRVN3 | Q63796, M3K12_RAT |
| P36004, KKQ8_YEAST | P09251, KR1_VZVD | O43283, M3K13_HUMAN |
| P36003, KKR1_YEAST | P13288, KR2_EBV | Q5R8X7, M3K13_PONPY |
| Q03533, KM8S_YEAST | P28966, KR2_EHV1B | Q99558, M3K14_HUMAN |
| P53739, KN8R_YEAST | P84391, KR2_EHV1V | Q9WUL6, M3K14_MOUSE |
| P53974, KNC0_YEAST | P04290, KR2_HHV11 | Q13233, M3K1_HUMAN |
| P32350, KNS1_YEAST | P30662, KR2_PRVN3 | P53349, M3K1_MOUSE |
| Q08217, KOE5_YEAST | P09296, KR2_VZVD | Q62925, M3K1_RAT |
| Q9Y7J6, KOIA_SCHPO | P54644, KRAC_DICDI | Q9Y2U5, M3K2_HUMAN |
| Q12236, KOK0_YEAST | Q07292, KRAF1_CAEEL | Q61083, M3K2_MOUSE |
| Q12222, KOM8_YEAST | P11346, KRAF1_DROME | Q99759, M3K3_HUMAN |
| Q9VPC0, KP58_DROME | O57259, KRB2_VACCA | Q61084, M3K3_MOUSE |
| Q19266, KPC3_CAEEL | P21098, KRB2_VACCC | Q9Y6R4, M3K4_HUMAN |
| P83099, KPC4_DROME | P24362, KRB2_VACCV | O08648, M3K4_MOUSE |
| Q99683, M3K5_HUMAN | Q10292, MEK1_SCHPO | O61443, MK14B_DROME |
| O35099, M3K5_MOUSE | P24719, MEK1_YEAST | P83100, MK14C_DROME |
| O95382, M3K6_HUMAN | Q14680, MELK_HUMAN | O02812, MK14_CANFA |
| Q9V3Q6, M3K7_DROME | Q61846, MELK_MOUSE | Q16539, MK14_HUMAN |
| O43318, M3K7_HUMAN | P43294, MHK_ARATH | P47811, MK14_MOUSE |
| Q62073, M3K7_MOUSE | Q23356, MIG15_CAEEL | Q95NE7, MK14_PANTR |
| P41279, M3K8_HUMAN | P00531, MIL_AVIMH | P70618, MK14_RAT |
| Q07174, M3K8_MOUSE | P46196, MK01_BOVIN | P47812, MK14_XENLA |
| Q63562, M3K8_RAT | P28482, MK01_HUMAN | P43068, MKC1_CANAL |
| P80192, M3K9_HUMAN | P63085, MK01_MOUSE | Q9BUB5, |
| Q5TCX8, M3KL4_HUMAN | P63086, MK01_RAT | MKNK1_HUMAN O08605, |
| Q95UN8, M3KSL_DROME | P26696, MK01_XENLA | MKNK1_MOUSE |
| P83104, M3LK7_DROME | P27361, MK03_HUMAN | Q9HBH9, MKNK2_HUMAN |
| Q92918, M4K1_HUMAN | Q63844, MK03_MOUSE | Q8CDB0, MKNK2_MOUSE |
| P70218, M4K1_MOUSE | P21708, MK03_RAT | Q9NYL2, MLTK_HUMAN |
| Q12851, M4K2_HUMAN | P31152, MK04_HUMAN | Q9ESL4, MLTK_MOUSE |
| Q61161, M4K2_MOUSE | Q6P5G0, MK04_MOUSE | Q07176, MMK1_MEDSA |
| Q8IVH8, M4K3_HUMAN | Q63454, MK04_RAT | Q40353, MMK2_MEDSA |
| Q99JP0, M4K3_MOUSE | Q16659, MK06_HUMAN | P49657, MNB_DROME |
| Q924I2, M4K3_RAT | Q61532, MK06_MOUSE | Q9UQ07, MOK_HUMAN |
| O95819, M4K4_HUMAN | P27704, MK06_RAT | Q9WVS4, MOK_MOUSE |
| P97820, M4K4_MOUSE | Q13164, MK07_HUMAN | P87347, MOS_APTAU |
| Q9Y4K4, M4K5_HUMAN | Q9WVS8, MK07_MOUSE | Q8QHF0, MOS_ATHNI |
| Q8BPM2, M4K5_MOUSE | Q90327, MK08A_CYPCA | Q8AX02, MOS_ATHSQ |
| Q8N4C8, M4K6_HUMAN | O42099, MK08B_CYPCA | P10650, MOS_CERAE |
| Q9JM52, M4K6_MOUSE | Q9DGD9, MK08_BRARE | P10741, MOS_CHICK |
| P20794, MAK_HUMAN | P45983, MK08_HUMAN | Q90XV8, MOS_CICNG |
| Q04859, MAK_MOUSE | Q91Y86, MK08_MOUSE | Q8AX01, MOS_DENAN |
| P20793, MAK_RAT | P49185, MK08_RAT | Q90XV6, MOS_GYMCA |
| Q8IW41, MAPK5_HUMAN | Q8QHK8, MK08_XENLA | P00540, MOS_HUMAN |
| O54992, MAPK5_MOUSE | P79996, MK09_CHICK | P00536, MOS_MOUSE |
| Q00859, MAPK_FUSSO | P45984, MK09_HUMAN | P07331, MOS_MSVMH |
| Q06060, MAPK_PEA | Q9WTU6, MK09_MOUSE | P00537, MOS_MSVMM |
| Q40884, MAPK_PETHY | P49186, MK09_RAT | P00538, MOS_MSVMO |
| Q9P0L2, MARK1_HUMAN | P53779, MK10_HUMAN | P32593, MOS_MSVMT |
| Q8VHJ5, MARK1_MOUSE | Q61831, MK10_MOUSE | P10421, MOS_MSVTS |
| O08678, MARK1_RAT | P49187, MK10_RAT | Q90XV9, MOS_NYCNY |
| Q7KZI7, MARK2_HUMAN | Q15759, MK11_HUMAN; | P50118, MOS_PIG |
| Q05512, MARK2_MOUSE | Q9WUI1, MK11_MOUSE | P00539, MOS_RAT |
| O08679, MARK2_RAT | O42376, MK12_BRARE | Q8AX00, MOS_SIBNE |
| P27448, MARK3_HUMAN | P53778, MK12_HUMAN | Q90XV7, MOS_VULGR |
| Q03141, MARK3_MOUSE | O08911, MK12_MOUSE | P12965, MOS_XENLA |
| Q96L34, MARK4_HUMAN | Q63538, MK12_RAT | P45985, MP2K4_HUMAN |
| Q9Y2H9, MAST1_HUMAN | O15264, MK13_HUMAN | P47809, MP2K4_MOUSE |
| Q9R1L5, MAST1_MOUSE | Q9Z1B7, MK13_MOUSE | O94235, MPH1_SCHPO |
| Q810W7, MAST1_RAT | Q9N272, MK13_PANTR | Q39021, MPK1_ARATH |
| Q6P0Q8, MAST2_HUMAN | Q9WTY9, MK13_RAT | Q39022, MPK2_ARATH |
| Q60592, MAST2_MOUSE | Q9DGE2, MK14A_BRARE | Q39023, MPK3_ARATH |
| Q96GX5, MASTL_HUMAN | Q90336, MK14A_CYPCA | Q39024, MPK4_ARATH |
| Q8C0P0, MASTL_MOUSE | O62618, MK14A_DROME | Q39025, MPK5_ARATH |
| P38615, MDS1_YEAST | Q9DGE1, MK14B_BRARE | Q39026, MPK6_ARATH |
| P38111, MEC1_YEAST | Q9I958, MK14B_CYPCA | Q39027, MPK7_ARATH |
| Q8AYG3, MPS1_BRARE | O75914, PAK3_HUMAN | P34206, PK1_ASFM2 |
| P54199, MPS1_YEAST | Q61036, PAK3_MOUSE | P41415, PK1_NPVAC |
| P50873, MRK1_YEAST | Q7YQL4, PAK3_PANTR | P41719, PK1_NPVHZ |
| Q8NEV4, | Q7YQL3, PAK3_PONPY | P41720, PK1_NPVLD |
| MYO3A_HUMAN | Q62829, PAK3_RAT | O10269, PK1_NPVOP |
| Q8K3H5, MYO3A_MOUSE | O96013, PAK4_HUMAN | Q9KIG4, PK1_STRTO |
| Q8WXR4, | Q8BTW9, PAK4_MOUSE | P41676, PK2_NPVAC |
| MYO3B_HUMAN O75011, | Q9NQU5, PAK6_HUMAN | Q9W0V1, PK61C_DROME |
| NAK1_SCHPO P43293, | Q9P286, PAK7_HUMAN | P54739, PKAA_STRCO |
| NAK_ARATH | Q9VXE5, PAKM_DROME | P54740, PKAB_STRCO |
| P84199, NEK1_CAEEL | Q96RG2, PASK_HUMAN | Q03407, PKH1_YEAST |
| Q96PY6, NEK1_HUMAN | Q8CEE6, PASK_MOUSE | Q16512, PKL1_HUMAN |
| P51954, NEK1_MOUSE | Q9FE20, PBS1_ARATH | P70268, PKL1_MOUSE |
| P51955, NEK2_HUMAN | Q00536, PCTK1_HUMAN | Q63433, PKL1_RAT |
| O35942, NEK2_MOUSE | Q04735, PCTK1_MOUSE | Q16513, PKL2_HUMAN |
| P51956, NEK3_HUMAN | Q63686, PCTK1_RAT | Q8BWW9, PKL2_MOUSE |
| Q9R0A5, NEK3_MOUSE | Q00537, PCTK2_HUMAN | O08874, PKL2_RAT |
| P51957, NEK4_HUMAN | Q8K0D0, PCTK2_MOUSE | P37562, PKN1_BACSU |
| Q9Z1J2, NEK4_MOUSE | O35831, PCTK2_RAT | Q822R1, PKN1_CHLCV |
| Q9HC98, NEK6_HUMAN | Q07002, PCTK3_HUMAN | Q9PKP3, PKN1_CHLMU |
| Q9ES70, NEK6_MOUSE | Q04899, PCTK3_MOUSE | Q7AJA5, PKN1_CHLPN |
| P59895, NEK6_RAT | Q5RD01, PCTK3_PONPY | O84147, PKN1_CHLTR |
| Q8TDX7, NEK7_HUMAN | O35832, PCTK3_RAT | Q8FUI5, PKN1_COREF |
| Q9ES74, NEK7_MOUSE | O15530, PDPK1_HUMAN | Q8NU98, PKN1_CORGL |
| Q90XC2, NEK8_BRARE | Q9Z2A0, PDPK1_MOUSE | P33973, PKN1_MYXXA |
| Q86SG6, NEK8_HUMAN | O55173, PDPK1_RAT | Q8R9T6, PKN1_THETN |
| Q91ZR4, NEK8_MOUSE | O74456, PEF1_SCHPO | O34507, PKN2_BACSU |
| Q8TD19, NEK9_HUMAN | O94921, PFTK1_HUMAN | Q97IC2, PKN2_CLOAB |
| Q8K1R7, NEK9_MOUSE | O35495, PFTK1_MOUSE | Q8XJL8, PKN2_CLOPE |
| Q7ZZC8, NEK9_XENLA | Q751E8, PHO85_ASHGO | Q8FUI4, PKN2_COREF |
| P48479, NIM1_NEUCR | Q9HGY5, PHO85_CANAL | Q8NU97, PKN2_CORGL |
| P10676, NINAC_DROME | Q6FKD4, PHO85_CANGA | P54736, PKN2_MYXXA |
| Q9UBE8, NLK_HUMAN | Q6BRY2, PHO85_DEBHA | Q9XBQ0, PKN3_MYXXA |
| O54949, NLK_MOUSE | Q92241, PHO85_KLULA | P54737, PKN5_MYXXA |
| O48963, NPH1_ARATH | Q6C7U8, PHO85_YARLI | P54738, PKN6_MYXXA |
| O42626, NRC2_NEUCR | P17157, PHO85_YEAST | Q8G4G1, PKNA2_BIFLO |
| Q08942, NRKA_TRYBB | Q9N0P9, PIM1_BOVIN | P54734, PKNA_ANASP |
| Q03428, NRKB_TRYBB | Q9YHZ5, PIM1_BRARE | P65727, PKNA_MYCBO |
| Q40517, NTF3_TOBAC | Q95LJ0, PIM1_FELCA | P54743, PKNA_MYCLE |
| Q40532, NTF4_TOBAC | P11309, PIM1_HUMAN | P65726, PKNA_MYCTU |
| Q40531, NTF6_TOBAC | P06803, PIM1_MOUSE | Q8G6P9, PKNB_BIFLO |
| O60285, NUAK1_HUMAN | P26794, PIM1_RAT | Q9CEF5, PKNB_LACLA |
| O13310, ORB6_SCHPO | Q9P1W9, PIM2_HUMAN | P0A5S5, PKNB_MYCBO |
| Q17850, PAK1_CAEEL | Q62070, PIM2_MOUSE | P54744, PKNB_MYCLE |
| Q13153, PAK1_HUMAN | Q9PU85, PIM3_COTJA | P0A5S4, PKNB_MYCTU |
| O88643, PAK1_MOUSE | Q86V86, PIM3_HUMAN | Q822K5, PKND_CHLCV; |
| P35465, PAK1_RAT | P58750, PIM3_MOUSE | Q9PK92, PKND_CHLMU |
| P38990, PAK1_YEAST | O70444, PIM3_RAT | Q9Z986, PKND_CHLPN |
| Q13177, PAK2_HUMAN | Q91822, PIM3_XENLA | O84303, PKND_CHLTR |
| Q8CIN4, PAK2_MOUSE | Q9BXM7, PINK1_HUMAN | O05871, PKND_MYCTU |
| Q29502, PAK2_RABIT | Q99MQ3, PINK1_MOUSE | Q7TZN3, PKNE_MYCBO |
| Q64303, PAK2_RAT | P42493, PK1_ASFB7 | P72001, PKNE_MYCTU |
| Q7TZN1, PKNF_MYCBO | Q922R0, PRKX_MOUSE | P70336, ROCK2_MOUSE |
| P72003, PKNF_MYCTU | O43930, PRKY_HUMAN | Q62868, ROCK2_RAT |
| P65729, PKNG_MYCBO | Q13523, PRP4B_HUMAN | P93194, RPK1_IPONI |
| P57993, PKNG_MYCLE | Q61136, PRP4B_MOUSE | P42411, RSBT_BACSU |
| P65728, PKNG_MYCTU | Q07538, PRP4_SCHPO | Q9K5J7, RSBW_BACAN |
| Q7U095, PKNH_MYCBO | Q96S44, PRPK_HUMAN | Q73CI0, RSBW_BACC1 |
| Q11053, PKNH_MYCTU | Q99PW4, PRPK_MOUSE | Q81H23, RSBW_BACCR |
| P65731, PKNI_MYCBO | Q12706, PSK1_SCHPO | Q63F14, RSBW_BACCZ |
| P65730, PKNI_MYCTU | Q9ZVR7, PSKR_ARATH | Q9KFF1, RSBW_BACHD |
| P65733, PKNJ_MYCBO | Q8LPB4, PSKR_DAUCA | Q6HMH0, RSBW_BACHK |
| P65732, PKNJ_MYCTU | P36002, PTK1_YEAST | O50231, RSBW_BACLI |
| Q7TXA9, PKNK_MYCBO | Q6FRE7, PTK2_CANGA | P17904, RSBW_BACSU |
| P95078, PKNK_MYCTU | P47116, PTK2_YEAST | Q92DC2, RSBW_LISIN |
| Q7TYY6, PKNL_MYCBO | Q9FKS4, RAD3A_ARATH | Q721S2, RSBW_LISMF |
| O53510, PKNL_MYCTU | Q02099, RAD3_SCHPO | Q8Y8K6, RSBW_LISMO |
| P47355, PKNS_MYCGE | P22216, RAD53_YEAST | Q8CXL7, RSBW_OCEIH |
| P75524, PKNS_MYCPN | P05625, RAF1_CHICK | Q5HED6, RSBW_STAAC |
| Q9XA16, PKNX_STRCO | P04049, RAF1_HUMAN | P0A0H6, RSBW_STAAM |
| Q01577, PKPA_PHYBL | Q99N57, RAF1_MOUSE | P0A0H7, RSBW_STAAN |
| Q9S2C0, PKSC_STRCO | P11345, RAF1_RAT | Q6GF08, RSBW_STAAR |
| P49695, PKWA_THECU | P09560, RAF1_XENLA | Q6G7P4, RSBW_STAAS |
| P34331, PLK1_CAEEL | P00532, RAF_MSV36 | P0A0H8, RSBW_STAAU |
| P53350, PLK1_HUMAN | P38622, RCK1_YEAST | Q8NVI5, RSBW_STAAW |
| Q07832, PLK1_MOUSE | P38623, RCK2_YEAST | Q9F7V2, RSBW_STAEP |
| Q62673, PLK1_RAT | P43565, RIM15_YEAST | Q75LR7, SAPK1_ORYSA |
| P70032, PLK1_XENLA | Q12196, RIO1_YEAST | Q84TC6, SAPK2_ORYSA |
| P62205, PLK1_XENTR | P40160, RIO2_YEAST | Q75V63, SAPK3_ORYSA |
| Q9N2L7, PLK2_CAEEL | Q9BRS2, RIOK1_HUMAN | Q5N942, SAPK4_ORYSA |
| Q9NYY3, PLK2_HUMAN | Q9BVS4, RIOK2_HUMAN | Q7XKA8, SAPK5_ORYSA |
| P53351, PLK2_MOUSE | Q9CQS5, RIOK2_MOUSE | Q6ZI44, SAPK6_ORYSA |
| Q9R012, PLK2_RAT | O14730, RIOK3_HUMAN | Q7XQP4, SAPK7_ORYSA |
| Q20845, PLK3_CAEEL | Q9DBU3, RIOK3_MOUSE | Q7Y0B9, SAPK8_ORYSA |
| Q9H4B4, PLK3_HUMAN | Q13546, RIPK1_HUMAN | Q75V57, SAPK9_ORYSA |
| Q60806, PLK3_MOUSE | Q60855, RIPK1_MOUSE | Q75H77, SAPKA_ORYSA |
| Q9R011, PLK3_RAT | O43353, RIPK2_HUMAN | P25333, SAT4_YEAST |
| O00444, PLK4_HUMAN | P58801, RIPK2_MOUSE | P11792, SCH9_YEAST |
| Q64702, PLK4_MOUSE | Q9Y572, RIPK3_HUMAN | P50530, SCK1_SCHPO |
| P50528, PLO1_SCHPO | Q9QZL0, RIPK3_MOUSE | P18431, SGG_DROME |
| Q17446, PMK1_CAEEL | Q9Z2P5, RIPK3_RAT | O00141, SGK1_HUMAN |
| Q8MXI4, PMK2_CAEEL | P57078, RIPK4_HUMAN | Q9WVC6, SGK1_MOUSE |
| O44514, PMK3_CAEEL | Q9LQQ8, RLCK7_ARATH | Q9XT18, SGK1_RABIT |
| O18209, PMYT1_CAEEL | P47735, RLK5_ARATH | Q06226, SGK1_RAT |
| Q9NI63, PMYT1_DROME | P27966, RMIL_AVEVR | Q9HBY8, SGK2_HUMAN |
| Q99640, PMYT1_HUMAN | P10533, RMIL_AVII1 | Q9QZS5, SGK2_MOUSE |
| Q9ESG9, PMYT1_MOUSE | Q8MIT6, ROCK1_BOVIN | Q8R4U9, SGK2_RAT |
| Q91618, PMYT1_XENLA | Q13464, ROCK1_HUMAN | Q96BR1, SGK3_HUMAN |
| P52304, POLO_DROME | P70335, ROCK1_MOUSE | Q9ERE3, SGK3_MOUSE |
| Q09690, POM1_SCHPO | P61584, ROCK1_PANTR | P23293, SGV1_YEAST |
| O13958, PRK1_SCHPO | O77819, ROCK1_RABIT | P50527, SHK1_SCHPO |
| P78527, PRKDC_HUMAN | Q63644, ROCK1_RAT | Q10056, SHK2_SCHPO |
| P97313, PRKDC_MOUSE | Q28021, ROCK2_BOVIN | O14305, SID1_SCHPO |
| P51817, PRKX_HUMAN | O75116, ROCK2_HUMAN | Q09898, SID2_SCHPO |
| Q12469, SKM1_YEAST | Q9UEE5, ST17A_HUMAN | P41895, T2FA_YEAST |
| Q12505, SKS1_YEAST | Q9GM70, ST17A_RABIT | P51123, TAF1_DROME |
| Q03656, SKY1_YEAST | O94768, ST17B_HUMAN | P21675, TAF1_HUMAN |
| Q00772, SLT2_YEAST | Q8BG48, ST17B_MOUSE | Q15569, TESK1_HUMAN |
| Q09488, SMA6_CAEEL | Q91XS8, ST17B_RAT | O70146, TESK1_MOUSE |
| P41808, SMK1_YEAST | Q9Y2H1, ST38L_HUMAN | Q63572, TESK1_RAT |
| P57059, SN1L1_HUMAN | Q7TSE6, ST38L_MOUSE | Q96S53, TESK2_HUMAN |
| Q60670, SN1L1_MOUSE | P23561, STE11_YEAST | Q8VCT9, TESK2_MOUSE |
| Q9R1U5, SN1L1_RAT | Q92212, STE20_CANAL | Q924U5, TESK2_RAT |
| Q9IA88, SN1L2_CHICK | Q03497, STE20_YEAST | P36897, TGFR1_HUMAN |
| Q9H0K1, SN1L2_HUMAN | P46599, STE7_CANAL | Q64729, TGFR1_MOUSE |
| Q8CFH6, SN1L2_MOUSE | P06784, STE7_YEAST | P80204, TGFR1_RAT |
| Q5REX1, SN1L2_PONPY | O94804, STK10_HUMAN | P37173, TGFR2_HUMAN |
| Q81MF4, SP2AB_BACAN | O55098, STK10_MOUSE | Q62312, TGFR2_MOUSE |
| Q731M3, SP2AB_BACC1 | Q15831, STK11_HUMAN | P38551, TGFR2_PIG |
| P70878, SP2AB_BACCO | Q91604, STK11_XENLA | P38438, TGFR2_RAT |
| Q819B3, SP2AB_BACCR | O75716, STK16_HUMAN | P34314, TLK1_CAEEL |
| Q635K7, SP2AB_BACCZ | O88697, STK16_MOUSE | Q9UKI8, TLK1_HUMAN |
| Q9KCN2, SP2AB_BACHD | P57760, STK16_RAT | Q8C0V0, TLK1_MOUSE |
| Q6HE93, SP2AB_BACHK | P49842, STK19_HUMAN | Q86UE8, TLK2_HUMAN |
| P26778, SP2AB_BACLI | Q9JHN8, STK19_MOUSE | O55047, TLK2_MOUSE |
| P35148, SP2AB_BACME | Q9UPE1, STK23_HUMAN | Q9UKE5, TNIK_HUMAN |
| O32724, SP2AB_BACSH | Q9Z0G2, STK23_MOUSE | P83510, TNIK_MOUSE |
| Q5WH26, SP2AB_BACSK | Q9Y6E0, STK24_HUMAN | Q6DHU8, TOPK_BRARE |
| O32727, SP2AB_BACST | Q99KH8, STK24_MOUSE | Q96KB5, TOPK_HUMAN |
| P10728, SP2AB_BACSU | O00506, STK25_HUMAN | Q9JJ78, TOPK_MOUSE |
| Q97GQ9, SP2AB_CLOAB | Q9Z2W1, STK25_MOUSE | Q9BX84, TRPM6_HUMAN |
| Q8XIR5, SP2AB_CLOPE | Q9BXU1, STK31_HUMAN | Q8CIR4, TRPM6_MOUSE |
| P59623, SP2AB_CLOTE | Q99MW1, STK31_MOUSE | Q96QT4, TRPM7_HUMAN |
| Q8EQ73, SP2AB_OCEIH | Q8TDR2, STK35_HUMAN | Q923J1, TRPM7_MOUSE |
| O32721, SP2AB_PAEPO | Q15208, STK38_HUMAN | Q9BXA7, TSSK1_HUMAN |
| P59624, SP2AB_PASPE | Q91VJ4, STK38_MOUSE | Q61241, TSSK1_MOUSE |
| Q8RAA8, SP2AB_THETN | Q9UEW8, STK39_HUMAN | Q96PF2, TSSK2_HUMAN |
| Q61IS6, SPK1_CAEBR | Q9Z1W9, STK39_MOUSE | O54863, TSSK2_MOUSE |
| Q03563, SPK1_CAEEL | O88506, STK39_RAT | Q96PN8, TSSK3_HUMAN |
| P27638, SPK1_SCHPO | Q13188, STK3_HUMAN | Q9D2E1, TSSK3_MOUSE |
| Q9FAB3, SPKA_SYNY3 | Q9JI10, STK3_MOUSE | Q6SA08, TSSK4_HUMAN |
| P74297, SPKB_SYNY3 | Q13043, STK4_HUMAN | Q9D411, TSSK4_MOUSE |
| P74745, SPKC_SYNY3 | Q91819, STK6L_XENLA | Q8TAS1, UHMK1_HUMAN |
| P54735, SPKD_SYNY3 | O14965, STK6_HUMAN | P97343, UHMK1_MOUSE |
| P73469, SPKF_SYNY3 | P97477, STK6_MOUSE | Q63285, UHMK1_RAT |
| Q92398, SPM1_SCHPO | P59241, STK6_RAT | O75385, ULK1_HUMAN |
| Q9UQY9, SPO4_SCHPO | Q91820, STK6_XENLA | O70405, ULK1 MOUSE |
| Q96SB4, SPRK1_HUMAN | P83098, STLK_DROME | Q23023, UNC51_CAEEL |
| O70551, SPRK1_MOUSE | Q9S713, STT7_ARATH | Q9J5B1, V111_FOWPV |
| Q5RD27, SPRK1_PONPY | Q84V18, STT7_CHLRE | Q9J523, V212_FOWPV |
| P78362, SPRK2_HUMAN | Q09892, STY1_SCHPO | Q9J509, V226_FOWPV |
| O94547, SRK1_SCHPO | P46549, SULU_CAEEL | Q03785, VHS1_YEAST |
| Q09092, SRK6_BRAOE | P39745, SUR1_CAEEL | O57252, VPK1_VACCA |
| O54781, SRPK2_MOUSE | Q05913, T2FA_DROME | P20505, VPK1_VACCC |
| P25390, SSK22_YEAST | P35269, T2FA_HUMAN | P16913, VPK1_VACCV |
| P50526, SSP1_SCHPO | Q04870, T2FA_XENLA | P33800, VPK1_VARV |
| P32216, VPK2_SWPVK | P83741, WNK1_MOUSE | Q09437, YP62_CAEEL |
| O57177, VPK2_VACCA | Q9JIH7, WNK1_RAT | Q11179, YPC2_CAEEL |
| P21095, VPK2_VACCC | Q9Y3S1, WNK2_HUMAN | P12688, YPK1_YEAST |
| P29884, VPK2_VACCP | Q9BYP7, WNK3_HUMAN | P18961, YPK2_YEAST |
| Q9JFE5, VPK2_VACCT | Q96J92, WNK4_HUMAN | Q9RI12, YPKA_YERPE |
| Q89121, VPK2_VACCV; | Q80UE6, WNK4_MOUSE | Q05608, YPKA_YERPS |
| P33801, VPK2_VARV | Q7TPK6, WNK4_RAT | Q20085, YPS7_CAEEL |
| Q9UVG6, VPS15_PICPA | Q58473, Y1073_METJA | Q09499, YQG4_CAEEL |
| P22219, VPS15_YEAST | Q8MYQ1, Y31E_CAEEL | Q09298, YQO9_CAEEL |
| Q7ZUS1, VRK1_BRARE | Q03021, Y396_THEAC | Q20347, YR62_CAEEL |
| Q99986, VRK1_HUMAN | Q57886, Y444_METJA | Q09595, YRL5_CAEEL |
| Q80X41, VRK1_MOUSE | P34516, YMX8_CAEEL | Q11090, YWY3_CAEEL |
| Q86Y07, VRK2_HUMAN | P45894, YNA3_CAEEL | Q621J7, ZYG1_CAEBR |
| Q8BN21, VRK2_MOUSE | P32742, YNH4_CAEEL | Q9GT24, ZYG1_CAEEL |
| Q8IV63, VRK3_HUMAN | P34633, YOO1_CAEEL | |
| Q8K3G5, VRK3_MOUSE | P34635, YOO3_CAEEL | |
| Q9H4A3, WNK1_HUMAN | P34649, YOT3_CAEEL | |

Analysis of each one of these enzymes, alone or in combination with others, is specifically contemplated in accordance with the teachings herein, as part of the invention.

Kinases associated with cancers include at least the following: Ab1 and BCR (BCR-Ab1 fusion, chronic myelogenous leukemia); Agc (within PI3-kinase signaling pathway; over-expressed in breast, prostate, lung, pancreatic, liver, ovarian, and colorectal cancers); Akt2 (amplified and over-expressed in ovarian and pancreatic tumors); Alk (lymphomas); Arg (differential expression in multiple cancers); Atm (loss-of-function mutations correlate with leukemias and lymphomas); Atr (stomach, endometrial cancers); AurA and AurB (amplified or overexpressed in many tumors); Axl (overexpressed in many cancers); B-Raf (melanoma and other cancers); Brk (breast and other cancers); BUB1 and BUBR1 (gastric and other cancers); Cdk1, Cdk2, Cdk4, and Cdk6 (activated in many cancers); Ck2 (lung and breast cancers); Cot/Tp12 (overexpressed in breast tumors); Ctk/MatK (breast cancer); DapK1; eEG2k (breast cancer); EGFR (over-expressed in head & neck and breast cancers); EphA1, EphA2, EphA3, EphB2, and EphB4 (multiple cancers); Fak (breast, ovarian, thyroid, other cancers); Fer (prostate); FGFR-1, FGFR-2, FGFR-3, and FGFR-4 (numerous cancers); Fgr (prostate); VEGFR-1, VEGFR-2, and VEGFR-3 (numerous cancers); mTOR (numerous cancers); FMS (breast and other cancers); Her-2, Her-3, and Her-4 (breast and other cancers); Hgk; HipK1 and HipK2; Ilk (increased expression in multiple tumors); Jak-1 and Jak-2; Kit (gastrointestinal stromal tumors); Lck (overexpressed in thymic tumors and other cancers); Met (numerous cancers); Mst4 (prostate cancer); NEK2 and NEK8; p38; Pak4 (overexpressed in several cancers); PDGFR-α and β; Pim1 (overexpressed in prostate cancer); Pim2 and Pim3; Pkc-α, Pkc-β, Pkc-δ, Pkc-ε, Pkc-η, and Pkc-θ (numerous cancers); Pkr; Plk1 (elevated expression in many cancers); Raf1 (amplified in many tumors); Ret; Ron (highly expressed in numerous cancers); p70s6k (elevated expression in colon and breast cancer); Src (increased expression and activity in numerous cancers); Syk (reduced expression in numerous cancers); TGFβR-1 and TGFβR-2; Tie2; TrkB; Tyro3; and Yes (amplification and/or increased expression in multiple cancers).

Kinases associated with cardiovascular disease or hypertension include Alk1, NPR1, BMPR2, CDK9, Erk5, Pkc-α, Pkc-δ, Pkc-ε, ROCK1 and ROCK 2, Tie 2, and Wnk1 and Wnk4.

Kinases associated with neurodegeneration, neurological, or central nervous system diseases include ATM (loss of function mutations associated with ataxia); CK1α, CK1δ, CK2α1 and CK2α2; DAPK1 (increased expression in epilepsy); DMPK1; Dyrk1a; Fyn (epilepsy); Gsk3α and GSK3β; Jnk3; Pak2; Pink1 (Parkinson's disease); PKcε (Alzheimer's disease); Pkcγ; Pkr; ROCK1 (Alzheimer's disease); and Rsk2.

The CDK9 kinase is associated with viral infection and replication, and inhibitors have been shown to block HIV replication and varicella zoster replication. Blockage of MEK1 and MEK2 appears to block export of influenza viral particles.

The FH4 receptor tyrosine kinase (VEGFR-3) has been associated with lymphangiogenesis and loss of function mutations associated with lymphedema.

Loss of function mutations in JAK3 are associated with severe combined immunodeficiency (SCID).

The enzymes that are evaluated using the disclosed methods may be involved in a signaling pathway. Signaling pathways include PI3K/AKT pathways; Ras/Raf/MEK/Erk pathways; MAP kinase pathways; JAK/STAT pathways; mTOR/TSC pathways; heterotrimeric G protein pathways; PKA pathways; PLC/PKC pathways; NK-kappaB pathways; cell cycle pathways (cell cycle kinases); TGF-beta pathways; TLR pathways; Notch pathways; Wnt pathways; Nutrient signaling pathways (AMPK signaling); cell-cell and cell:substratum adhesion pathways (such as cadherin, integrins); stress signaling pathways (high/low salt, heat, radiation); cytokine signaling pathways; antigen receptor signaling pathways; and co-stimulatory immune signaling pathways. In some cases, the methods may be used to measure the activity of more than one enzyme involved in the same signaling pathway. Numerous resources are widely known with descriptions of pathways, including www.biocarta.com, www.cellsignal.com, and www.signaling-gateway.org.

Enzymatic activity is measured by MS detection of an enzyme's substrate and/or reaction product. In one exemplary embodiment, a sample containing (or suspected of containing) one or more enzymes of interest and in the presence of a plurality of enzymes is contacted with a substrate composition. The substrate composition contains a substrate specific for the enzyme of interest and, as necessary, other reagents, buffers, salts, and/or cofactors required or preferred to allow the enzymatic reaction to occur on the substrate in order to form a product.

The substrate is transformed in this enzymatic reaction to a product of known mass. In one embodiment, the enzyme of interest is a kinase, such as a kinase as listed in Table 1, and the substrate of interest is a peptide substrate, such as those listed in Table 2. Specific substrate peptides for protein kinases have been identified through a variety of means, for example, in Benton et al., Curr Proteomics, 1(2):8-120 (2004), incorporated herein in its entirety by reference. Many commercial sources exist for specific peptide substrates for protein kinases. Examples include but are not limited to Sigma (St. Louis, Mo. USA) and BIAFFIN GmbH & Co KG (Kassel Germany). The peptide substrate is modified in the presence of the appropriate kinase and ATP to form a phosphorylated peptide product, as listed in Table 2. It will be appreciated from the description herein that knowledge of which residue is phosphorylated is not critical to practice of the invention. One only needs to know the mass for MS using a single analyzer. For tandem MS, it is useful to know where the modification on the substrate occurs and the masses of the fragment ions.

TABLE 2

| | | | Peptide | | |
|---|---|---|---|---|---|
| | | | Product | | |
| | | | (pX = | | |
| Enzyme or | | | phosphorylated | | |
| Enzyme | Peptide | SEQ ID | amino acid | SEQ ID | Pathway |
| Family | Substrate | NO. | residue) | NO. | involved |
| Casein | RRR DDD | SEQ ID | RRR DDD | SEQ ID | Wnt signaling |
| Kinase 2 | SDD D | NO: 1 | pSDD D | NO: 33 | |
| IKK | KKK KER | SEQ ID | KKK KER LLD | SEQ ID | NFkappaB |
| | LLD DRH | NO: 2 | DRH DSG | NO: 34 | |
| | DSG LDS | | LDpS MKD EE | | |
| | MKD EE | | | | |
| JNK3 | RRE LVE | SEQ ID | RRE LVE PLT | SEQ ID | MAP kinase |
| | PLT PSG | NO: 3 | PpSG EAP | NO:35 | |
| | EAP NQA | | NQA LLR | | |
| | LLR | | | | |
| PKC | ERM RPR | SEQ ID | ERM RPR KRQ | SEQ ID | Calcium |
| | KRQ GSV | NO: 4 | GpSV RRR V | NO: 36 | signaling |
| | RRR V | | | | |
| S6 Kinase/ | RRR LSS | SEQ ID | RRR LpSS | SEQ ID | Growth |
| Rsk | LRA | NO: 5 | LRA | NO: 37 | factor, insulin |
| | | | | | and PI3K/Akt |
| Abl | EAI YAA | SEQ ID | EAI pYAA PFA | SEQ ID | Growth factor |
| | PFA KKK | NO: 6 | KKK | NO: 38 | |
| Akt | RPR AAT F | SEQ ID | RPR AApT F | SEQ ID | Growth |
| | | NO: 7 | | NO: 39 | factor, insulin |
| | | | | | and PI3K/Akt |
| GSK3 | RRR PAS | SEQ ID | RRR PApS VPP | SEQ ID | Growth |
| | VPP SPS | NO: 8 | SPS LSR HSS | NO: 40 | factor, insulin |
| | LSR HSS | | HQR R | | and PI3K/Akt |
| | HQR R | | | | |
| IGF-1R | KKK SPG | SEQ ID | KKK SPG | SEQ ID | Growth factor |
| | EYV NIE FG | NO: 9 | EpYV NIE FG | NO: 41 | |
| MAP | APR TPG | SEQ ID | APR pTPG | SEQ ID | Growth |
| Kinase | GRR | NO: 10 | GRR | NO: 42 | factor, insulin |
| | | | | | and MAP |
| | | | | | kinase |
| | | | | | pathways |
| PKA | ISN RRG | SEQ ID | ISN RRG pTRG | SEQ ID | Heterotrimetic |
| | TRG | NO: 11 | | NO: 43 | G protein and |
| | | | | | protein kinase |
| | | | | | A signaling |
| PKC | QKR PSQ | SEQ ID | QKR PpSQ | SEQ ID | Calcium |
| | RSK YL | NO: 12 | RSK YL | NO: 44 | signaling |
| S6/Rsk | KKR NRT | SEQ ID | KKR NRpT | SEQ ID | Growth |
| | LTK | NO: 13 | LTK | NO: 3745 | factor, insulin |
| | | | | | and PI3K/Akt |
| Src | KVE KIG | SEQ ID | KVE KIG EGT | SEQ46 ID | Growth factor |
| | EGT YGV | NO: 14 | pYGV VYK | NO: | |
| | VYK | | | | |
| Akt-2T | ARK RER | SEQ ID | ARK RER | SEQ ID47 | Growth factor, |
| | TYS FGH | NO: 15 | pTYS FGH HA | NO: | insulin and |
| | HA | | | | PI3K/Akt |
| Ca²⁺ | SSV SLT | SEQ ID | SSV pSLT RSL | SEQ ID | Calcium |
| calmodulin- | RSL P | NO: 16 | P | N48O: | signaling |
| depedendent | | | | | |
| protein | | | | | |
| kinase II | | | | | |
| Casein | RRK DLH | SEQ ID | RRK DLH DDE | SEQ ID | Wnt signaling |
| kinase I | DDE EDE | NO: 17 | EDE AMpS | NO: 49 | |
| | AMS ITA | | ITA | | |
| Casein | RRA DDS | SEQ ID | RRA DDpS | SEQ ID | Wnt signaling |
| kinase II | DDD D | NO: 18 | DDD D | NO: 50 | |
| Cyclin- | HAT PPK | SEQ ID | HApT PPK | SEQ ID | Cell cycle |
| dependent | KKR K | NO: 19 | KKR K | NO: 51 | control |
| protein | | | | | |
| kinase 1 | | | | | |
| Cyclin- | PKT PKK | SEQ ID | PKpT PKK | SEQ ID | Cell cycle |
| dependent | AKK L | NO: 20 | AKK L | NO: 52 | control |
| protein | | | | | |
| kinase 5 | | | | | |
| GSK-3b | GPH RST | SEQ ID | GPH RpST PES | SEQ ID | Growth factor, |
| | PES RAA V | NO: 21 | RAA V | NO: 53 | insulin and |
| | | | | | PI3K/Akt |
| p44MAPK | APR TPG | SEQ ID | APR pTPG | SEQ ID | Growth factor, |
| (ERK1) & | GRR | NO: 22 | GRR | NO: 54 | insulin and |
| p42MAPK | | | | | MAP kinase |
| (ERK2) | | | | | pathways |
| Protein | RRG RTG | SEQ ID | RRG RpTG | SEQ ID | Calcium |
| Kinase C | RGR RGI FR | NO: 23 | RGR RGI FR | NO: 55 | signaling |
| Protein | QKR PSQ | SEQ ID | QKR PpSQ | SEQ ID | Calcium |
| Kinase C | RSK YL | NO: 24 | RSK YL | NO: 56 | signaling |
| Tyrosine | RR LIED | SEQ ID | RR LIED AEpY | SEQ ID | Growth factor |
| Kinase | AEY AAR G | NO: 25 | AAR G | NO: 57 | and insulin |
| Protein | LRR WSL G | SEQ ID | LRR WpSL G | SEQ ID | Heterotrimetic |
| Kinase A | | NO: 26 | | NO: 58 | G protein and |
| | | | | | protein kinase |
| | | | | | A signaling |
| MAP | KRE LVE | SEQ ID | KRE LVE PLT | SEQ ID | Growth factor, |
| Kinase | PLT PSG | NO: 27 | PpSG EAP | NO: 59 | insulin and |
| | EAP NQA | | NQA LLR | | MAP kinase |
| | LLR | | | | pathways |
| MAP | ADP DHD | SEQ ID | ADP DHD | SEQ ID | Growth factor, |
| Kinase | HTG FLT | NO: 28 | HpTG FLT | NO: 60 | insulin and |
| | EYV ATR | | EYV ATR | | MAP kinase |
| | WRR | | WRR | | pathways |
| MAP | KGA EAV | SEQ ID | KGA EAV | SEQ ID | Growth factor, |
| Kinase | TSP R | NO: 29 | pTSP R | NO: 61 | insulin and |
| | | | | | MAP kinase |
| | | | | | pathways |
| p21 | AKR ESA A | SEQ ID | AKR EpSA A | SEQ ID | Cell cycle |
| activated | | NO: 30 | | NO: 62 | control |
| protein | | | | | |
| kinase | | | | | |
| p38 | KKL RRT | SEQ ID | KKL RRT | SEQ ID | Cell cycle |
| activated | LSV A | NO: 31 | LpSV A | NO: 63 | control |
| protein | | | | | |
| kinase | | | | | |
| AMP | HMR SAM | SEQ ID | HMpR SAM | SEQ ID | Nutrient and |
| dependent | SGL HLV | NO: 32 | SGL HLV KRR | NO: 64 | energy |
| kinase | KRR | | | | sensing |
| | | | | | pathways |

The peptides listed in this table as suitable substrates are exemplary only. Many enzymes that can operate on a substrate of, e.g., ten amino acids as set forth in the table also can operate (1) on a longer substrate that includes the ten amino acids at the N-terminus, C-terminus, or middle of the longer substrate; (2) a shorter substrate than the ten residues listed in the table; (3) a substrate with sequence variation from the substrate in the table, and longer or shorter variations thereof.

Because a specific peptide substrate of unique mass can be selected or designed for multiple enzymes, the activity of more than one enzyme kinase may be measured and evaluated in one sample preparation. For example, a sample may contain both the kinases PKA and Akt, each of which has a specific peptide substrate (SEQ ID NO: 11 and SEQ ID NO: 7, respectively). Addition of both peptide substrates and appropriate co-reagents into the sample independently starts each enzymatic reaction. Aliquots may be collected at various time points, or only once, and analyzed using MS, wherein each enzyme's peptide substrate and product correlates to unique signals in the MS spectrum. Measurements of different kinds of enzymes may also be measured using the disclosed methods, such as, for example, combinations of two or more of any of kinase, transferase, hydrolase, lyase, isomerase, and/or ligase.

In general, reagents are added included in a sample and/or substrate to prevent enzyme or substrate degradation (e.g., protease inhibitors); preserve enzymatic activity (e.g, buffers, temperature, co-factors, salt concentration, ionic strength, pH, energy sources, and co-reagents); and prevent degradation of enzymatic reaction product (e.g., phosphatase inhibitors to prevent degradation of reaction products of kinases). With respect to preservation of enzymatic activity, prior literature that reports studies of enzymatic activity provides a rich source for information about buffers, pH, temperature, and other reaction conditions that are suitable for the same or similar enzymes for practicing methods of this invention. More generally, conditions that mimic an enzyme's natural environment (e.g., physiological temperature, pH, and ionic strength for many human or animal enzymes) are suitable for the present invention. Nonlimiting examples of reagents, buffers, salts, cofactors, inhibitors, include adenosine triphosphate (ATP), magnesium chloride, sodium chloride, phosphate buffers, iron, protease inhibitors, phosphatase inhibitors, Tris-HCl, HEPES, and chelating agents.

Exemplary protease inhibitors include, but are not limited to Na-p-tosyl-L-lysine chlormethyl ketone hydrochloride (TLCK), phenylmethylsulphonylfluoride (PMSF), leupeptin, pepstatin A, aprotinin, 4-(2-aminoethyl)benzenesulfonylfluoride hydrochloride (AEBSF), 6-aminohexanoic acid, antipain hydrochloride {[(S)-1-carboxy-2-phenylethyl]-carbamoyl-L-arginyl-L-valyl-arginal-phenylalanine}, benzamidine hydrochloride hydrate, bestatin hydrochloride, chymostatin, epoxysuccinyl-L-leucyl-amido-(4-guanidino)butane, ethylenediamine tetraacetic acid disodium salt, N-ethylmaleimide, and Kunitz trypsin inhibitor.

Exemplary phosphatase inhibitors include, but are not limited to, sodium fluoride, sodium orthovanadate, ocadaic acid, Vphen, microcystin, b-glycerophosphate, lacineurin, cantharidic acid, cyclosporin A, delamethrin, dephostatin, endothall, fenvalerate, fostriecin, phenylarsine oxide, and resmethrin.

The contacting of the enzyme and substrate, e.g., by the addition of the substrate to the biological sample (and, as appropriate, addition of other reagents and inhibitors) starts the enzymatic reaction. The reaction mixture is brought to a temperature sufficient to allow the enzymatic reaction to occur. This temperature can be between 0° C. and 100° C., more preferably, 0-75° C. or 0-50° C. In certain cases, the temperature is in the range of about 35° C. and 40° C. In some cases, the temperature is physiological temperature, or about 37° C. Other temperatures contemplated include about 20, 21, 22, 23, 24, 25, 26, 27, 28, 29, 30, 31, 32, 33, 34, 35, 36, 37, 38, 39, 40, 41, 42, 43, 44, and 45° C. The pH of the reaction mixture is also adjusted to a pH sufficient to allow the enzymatic reaction to occur. The pH may be in the range of about 0 to 14, and more preferably, about 5 to about 9, or about 6 to about 8. In some cases, the pH is about 7.4.

The reaction mixture is allowed to react for at least a time sufficient to produce enough reaction product to be measured by the analytical machines. In some variations, aliquots are collected at different time points to assess the rate of the reaction, while in others, only one aliquot at one time point is collected. The length of time that the enzymatic reaction occurs will be dependent upon the enzyme of interest, its concentration and activity in the sample, and in the purposes of the measurements, and will be easily determined by the person of skill in the art, in view of this disclosure.

Aliquots may be collected over a period time or one aliquot may be collected for a single analysis for a sample. The number of product molecules produced in an enzymatic reaction is dependent upon the incubation time. Therefore, the concentration or amount of product formed by the enzyme of interest may be normalized to the incubation time, which would allow for comparisons between data sets, time points, or samples. In some cases, the units of measurement for amount of product formed for an enzyme of interest are amount of product formed per unit time normalized to enzyme or lysate amounts (e.g., mol/s/Kg or pmol/min/mg).

One or more internal standards may be added to each aliquot to allow for quantification of product formed in each enzymatic reaction. Internal standards include, but are not limited to, isotopically labeled peptides, and compound structurally related to the product or substrate to be quantified. In some cases, only one internal standard is added; in other cases, two or more internal standards are added. In one embodiment, an internal standard is added for each enzymatic reaction of interest, wherein each internal standard is an isotopically labeled peptide product of the enzyme.

Isotopically labeled peptides are peptides that incorporate at least one rare isotope atom, such as a ¹³C, ¹⁵N, and/or ²H atom, so as to give the labeled peptide an essentially identical molecular structure but different molecular weight than the substrate or product. Stable isotopes (non-decaying isotopes or isotopes with very long half lives) are preferred, and among isotopes that do decay, those that decay to give off lower level radiation are preferred. Incorporation of one or more isotopes can be accomplished in a variety of ways. Amino acids containing one or more ¹³C, ¹⁵N and/or ²H can be obtained from commercial sources such as Sigma-Aldrich (Milwaukee, Wis., USA) and, using a peptide synthesizer, these isotopically labeled amino acids can be integrated into a peptide sequence. Isotopically labeled peptides can be produced by recombinant DNA techonology. Organisms such as bacteria are transfected with a plasmid bearing a sequence for a peptide that may be an internal standard. By growing bacteria in media in which one amino acid is replaced by its isotopically labeled counterpart, it is possible to obtain the labeled peptide using standard purification methods. Such methods are described in U.S. Pat. No. 5,885,795 and U.S. Pat. No. 5,151,267, each of which is incorporated by reference in its entirety.

The aliquot from the enzymatic reaction, including the internal standard, is then analyzed using a mass spectrometer. The aliquot may optionally be subjected to a purification step prior to MS analysis. Such purification includes, but is not limited to, liquid chromatography such as reverse phase, normal phase, ion exchange or size exclusion chromatography; filtration; solid phase extraction; solvent extraction; precipitation, and the like.

MS analysis involves the measurement of ionized analytes in a gas phase using an ion source that ionizes the aliquot, a mass analyzer that measures the mass-to-charge (m/z) ratio of the ionized aliquots, and a detector that registers the number of ions at each m/z value. The MS apparatus may be coupled to separation apparatus (e.g., such as chromatography columns, on-chip separation systems, and the like) to improve the ability to analyze complex mixtures.

Tandem MS (interchangeably called MS/MS herein) analysis involves a gas phase ion spectrometer that is capable of performing two successive stages m/z-based discrimination of ions in an ion mixture. This includes spectrometers having two mass analyzers as well as those having a single mass analyzer that are capable of selective acquisition or retention of ions prior to mass analysis. These include ion trap mass spectrometers, ion trap-TOF mass spectrometers, TOF-TOF mass spectrometers, triple quadrupoles, quadrupole-TOF (Q-TOF), and Fourier transform ion cyclotron resonance mass spectrometers.

A range of ions with different mass-to-charge (m/z) values can be trapped simultaneously in a quadrupole ion trap by the application of a radio frequency (RF) voltage to the ring electrode of the device. The trapped ions all oscillate at frequencies that are dependent on their m/z, and these frequencies can be readily calculated. Tandem MS is then performed by carrying out three steps. First, the analyte ions having the single m/z of interest (parent ions) are isolated by changing the RF voltage applied to the ring electrode and by applying waveforms (i.e. appropriate ac voltages to the endcap electrodes) with the appropriate frequencies that resonantly eject all the ions but the m/z of interest. Second, the isolated parent ions are then resonantly excited via the application of another waveform that corresponds to the oscillation frequency of the parent ions. In this way, the parent ions' kinetic energies are increased, and they undergo energetic collisions with the background gas (usually helium), which ultimately result in their dissociation into product ions. Third, these product ions are then detected with the usual mass analysis techniques in MS.

Multiplexed MS/MS refers to measuring the activity of several enzymes within the same assay. Multiple reaction monitoring (MRM) may be used for multiplexed MS/MS analysis, wherein MRM is performing several MS/MS measurements simultaneously on ions of multiple m/z ratios.

In some variations, collision induced dissociation (CID) may be employed during MS analysis. CID is a mechanism by which to fragment molecular ions in the gas phase. The molecular ions are usually accelerated by some electrical potential to high kinetic energy in the vacuum of a mass spectrometer and then allowed to collide with neutral gas molecules (often helium, nitrogen or argon). In the collision some of the kinetic energy is converted into internal energy which results in bond breakage and the fragmentation of the molecular ion into smaller fragments. These fragment ions can then be analyzed by a mass spectrometer. CID and the fragment ions produced by CID are used for several purposes. By looking for a unique fragment ion, it is possible to detect a given molecule in the presence of other molecules of the same nominal molecular mass, essentially reducing the background and increasing the limit of detection.

When the activity of more than one enzyme is measured, a mass spectrometer can be set up so that it analyzes individually each peptide product/internal standard combination. This can be accomplished using tandem MS analysis, wherein the sample is may be fractioned into a specific mass range, correlating with the substrate and/or product of a first enzyme, and separated from the rest of the sample, and then the specified molecules are broken into fragments and analyzed for amount of product formed by the first enzyme. A fraction having a different mass range can then be isolated from the same sample with the second mass range, correlating with a second enzyme's substrate and/or product, and analyzed. The means of doing multiple analyses of analytes by tandem mass spectrometry are described, for example, in U.S. Pat. Nos. 5,206,508; 6,649,351; 6,674,096; and 6,924,478, each of which is incorporated in its entirety by reference.

The MS analysis results in a spectrum of ion peaks with relative intensities relating to their concentration in the aliquot. When an internal standard of known quantity or concentration and volume is added to the sample, the relative signal strengths of the peptide internal standard peak and product peak may be calculated to give an enzyme activity in relative terms. Multiplication of the ratio of signal strengths between the internal standard and peptide product with the known concentration of the standard yields a quantitative measurement of the product, which in turn represents a quantitative measurement of the activity of the enzyme. For example, if the ratio of peptide product to internal standard is 1:0.5, the concentration of the peptide product will be two times the concentration of the internal standard. In variations where more than one enzyme is being evaluated, each enzyme's activity can be assessed by the same means of measuring the ratio of the first enzyme's product peak to its internal standard and independently, the ratio of the second enzyme's product peak to its internal standard.

Since the enzyme activity can be given in absolute terms, the enzymatic activity of particular enzymes can be compared from sample to sample, allowing for the assessment of enzymatic activity from one sample, or patient, to another; or from one treatment to another. This may allow for the rapid diagnosis of a particular diseases state or for the assessment of the efficacy of a particular treatment in view of a different treatment.

The methods described herein may be used to assess or screen an organism, human, or animal subject for abnormalities by detecting aberrant enzyme activity. By understanding the connection between specific enzymes and disease states, the methods allow for rapid determination of one or more enzyme activities which may be correlated to specific disease states. In some cases, more than one aberrant enzyme may be detected. By collecting samples from an organism or subject of interest and applying that sample to the methods disclosed herein one may be able to diagnose or screen for abnormalities which may then be linked to specific disease states. The aberrant enzyme activity may be detected by comparing the enzyme activity of the sample from the organism with a reference sample. Reference samples may be from the same organism at a different time or from a different location in the organism, or may be from a different organism of the same species, or a statistical measurement calculated from measurements of samples of cells of the same cell type, from multiple organisms of the same species, to provide an average for that organism and that cell type.

Another aspect of the invention is a kit for practicing the disclosed methods. Such kits may include (1) a plurality of substrate containers, where the substrate containers contain at least one substrate for an enzyme which can be modified in the presence of that enzyme to form a product, and (2) a plurality of standard containers, where the standard containers contain at least one mass labeled standard of a known concentration, where the mass labeled standard is identical to one of the products but has a different molecular weight from that of the product, due to inclusion of one or more isotopes into either the standard or the product. The substrates in these containers, in some cases, may be peptide substrates for enzymes which have 5 to 250 amino acid residues, and more preferably, 6 to 45 amino acid residues. The kits may also include one or more containers that hold additional reagents useful for practicing methods of the invention, such as a container which has protease inhibitors.

In some cases, the kit may include containers containing a composition of a mixture of two or more standards having a known molecular weight and concentration, where each standard is structurally identical to an enzyme product and has a molecular weight different than the enzyme product due to incorporation of at least one isotopic label in the standard. Preferred isotopic labels are those that are stable (e.g., long half-life and/or do not undergo significant radioactive decay), and that are rare (e.g., negligible amounts occurring in natural biomolecules).

Additional aspects and details of the disclosure will be apparent from the following examples, which are intended to be illustrative rather than limiting.

### EXAMPLES

### Example 1

The following example demonstrates that the amount of product of an enzymatic reaction can be quantified by comparison of the product peak and an internal standard peak using MS.

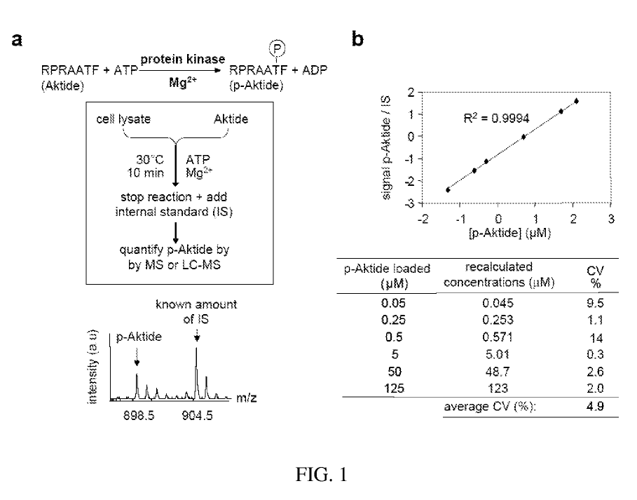

Six different samples were created of varying concentrations of a phosphorylated peptide product (SEQ ID NO: 39) generated by Akt: 0.05, 0.25, 0.5, 5, 50, and 125 μM. To each sample was added 0.5 μM of a mass labeled peptide product as an internal standard. This internal standard was 6 Daltons heavier than the peptide product. This internal standard was prepared by synthesizing a peptide with the same sequence as the product of the reaction but replacing standard L-proline with isotopically labeled L-proline (¹³C and ¹⁵N). The samples were each analyzed using a MALDI-TOF MS machine (Ultraflex, Bruker) or LC-MS/MS (Ultimate HPLC, Dionex, connected to a Micromass/Waters Q-TOF instrument) using an nano-electrospray ionization (nanoESI) interface. The analysis was performed by monitoring the parent-daughter ion transition of m/z 449.7 to m/z 400.3 for the peptide product and 452.7 to 403.3 for the internal standard. Reaction products were analyzed by LC-MS/MS without further treatment. Samples for MALDI-TOF MS analysis were prepared by solid phase extraction using a modified ZipTip™ (Millipore) or by strong cation exchange over a polysulphoethyl A resin (PolyLC, USA). The graph of FIG. 1B shows the correlation between the ratio of the peak intensities of peptide product to internal standard to concentration of peptide product. The recalculated concentrations of amount of peptide product in each sample was within ˜5% variation from the known amount (FIG. 1B, lower), indicating that this method of analysis accurately produces quantitative measurements of the peptide product.

### Example 2

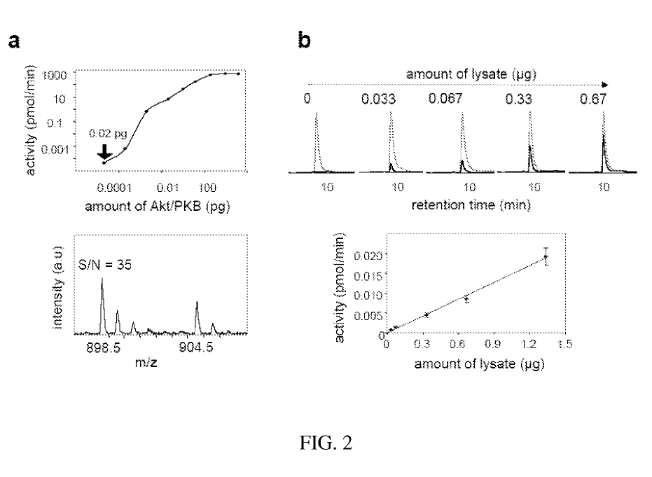

The activity of recombinant Akt/PKB was measured using the methods of the invention. Recombinant Akt/PKB was purchased from Upstate (Hampshire, UK). A 5.0 μL aliquot of 150 mM ATP, 150 mM substrate (SEQ ID. NO: 7), 7.5 mM magnesium chloride, 0.15 mM EDTA, 7.5 mM β-glycerol phosphate, 0.1 mM sodium orthovanadate, and 0.1 mM DTT was mixed with 2.5 μL solution of Akt/PKB of various amounts: 0.004, 0.04, 0.2, 0.8, 4, 20, and 80 ng. The incubation time of each reaction was from between 2 minutes to 18 hours. Reactions were stopped with the addition of 7.5 μL of a 1% trifluoroacetic acid solution containing 1 pmol/μL internal standard (mass labeled SEQ ID NO: 39). Aliquots (0.5 μL out of a total of 20 μL) from each reaction were analyzed by either MALDI-TOF MS (Ultraflex, Bruker) or LC-MS/MS (Ultimate HPLC, Dionex, connected to a Micromass/Waters Q-TOF instrument) using a nanoESI interface. Reaction products were analyzed by LC-MS/MS without further treatment. Samples for MALDI-TOF MS analysis were prepared by solid phase extraction using a modified ZipTip™ (Millipore) or by strong cation exchange over a polysulphoethyl A resin (PolyLC, USA). The analysis was performed by monitoring the parent-daughter ion transition of m/z 449.7 to m/z 400.3 for the peptide product and 452.7 to 403.3 for the internal standard. FIG. 2A shows that the MS analysis using the disclosed methods was capable of detecting enzymatic activity down to the zeptommole range.

### Example 3

Mouse WEHI-231 B lymphoma cell line was cultured as described in Cutillas et al., Mol Cell Proteomics 4:1038-51 (2005), incorporated herein in its entirety by reference. Cells were stimulated with anti-IgM (1 μM, 5 minutes) or pervanadate (500 μM, 30 minutes). Cells were treated with PI3K inhibitors for 30 minutes prior to lysis. Cultured cells were lysed in lysis buffer (1% Triton X100, 150 mM NaCl, 1 mM EDTA, Tris.HCl pH 7.4, 1 mM DTT, containing protease and phosphatase inhibitors). After centrifugation at 20,000×g, cell lysates were ready to use as enzyme sources. The enzyme activity of Akt in varying amounts of cell lysate (0.033, 0.067, 0.33, and 0.67 μg) was measured in the protocol outlined in example 2. The incubation time of each enzymatic reaction was between 2 and 10 min at 30° C. FIG. 2B shows the correlation between amount of lysate and measured activity of Akt.

### Example 4

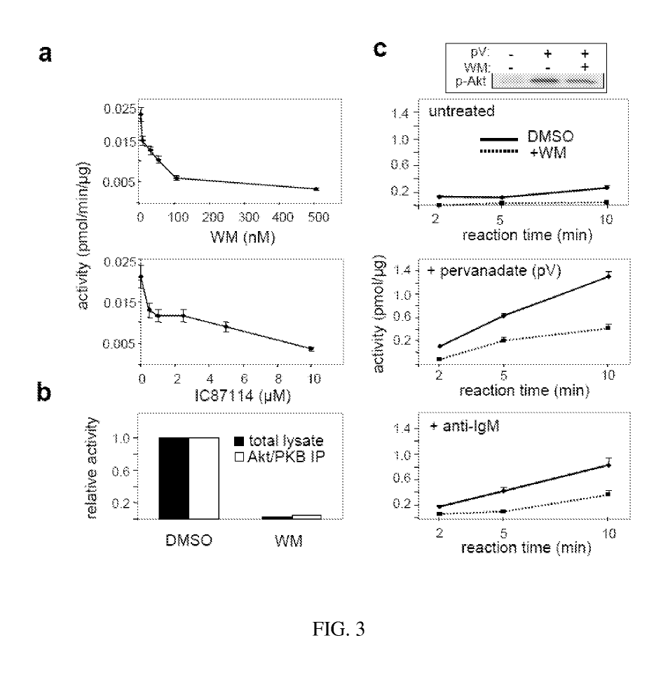

The sensitivity of the B lymphoma cell line to pre-treatment with PI3K inhibitors was assessed. Activity of the Akt enzyme in B lymphoma cell line WEHI-231 in the cell lysates was measured in the presence of varying concentrations of the PI3K inhibitors WM and IC87114 (FIG. 3A), indicating that the disclosed methods can accurately measure decreased enzyme activity. Here, each experiment was performed as described in example 2, above, but varying concentrations of each inhibitor was added to the reaction mixture. Activity of Akt immunoprecipitates were similarly affected by the addition of the PI3K inhibitor WM as measured by the disclosed methods (FIG. 3B).

### Example 5

Activity of Akt in B cell lysates was measured in the presence of PI3K activators (sodium pervanadate and anti-IgM), both in the presence and in the absence of WM. As seen in FIG. 3C, the activity measurements as obtained using the disclosed methods were sensitive to the presence or absence of the inhibitor WM.

### Example 6

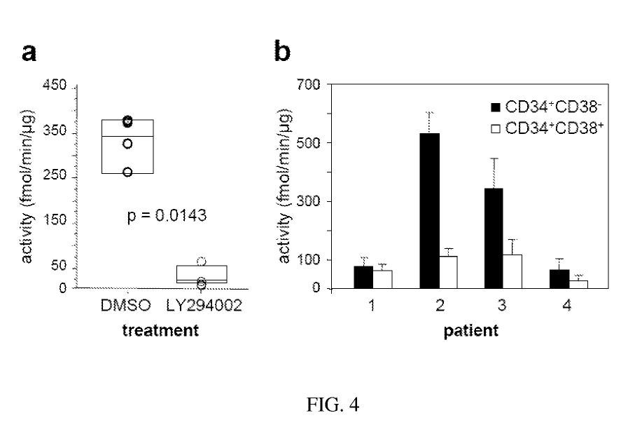

The activity of Akt in solid tumors was assessed from mouse B16 melanoma tumor biopsies. Seven days after intradermal injection of 2×10⁵ B16/B16 melanoma cells into mice, tumors were injected with 50 μL of 10 μM LY294002, a PI3K inhibitor, or a vehicle 2 hours before surgical excision. The samples were then analyzed using the protocol as described in example 2. FIG. 4A shows the activity measurements obtained, wherein the activity is drastically different for the samples from the vehicle treated tumors and the PI3K inhibitor treated tumors, indicating that the methods are highly specific for measuring the activity of the enzyme of interest (here, Akt).

### Example 7

The sensitivity of the disclosed methods also allowed quantification of Akt activity in the rare cancer stem cell populations. Relatively small numbers of these cells can be isolated routinely, and this limitation of number of cells has precluded the use of standard biochemical assays.

Samples from four patients having acute myeloid lymphoma (AML) were collected and snap frozen. Frozen primary samples were rapidly thawed, washed, and allowed to recover in RPMI 1640/10% FCS at 37 C, 5% CO₂ for 3 hours. Cells were then incubated with anti-CD34 (PE-conjugated, BD-Pharmingen) and anti-CD38 (FITC-conjugated, Dako) monoclonal antibodies for 30 minutes on ice. Cells were sorted in phosphate buffered saline into CD34⁺38⁻ (stem cell) and CD34⁺38⁺ fractions on an SPICS-Elite flow cytometer (Beckman-Coulter). After centrifugation, cell pellets were re-suspended in RPMI 1640/10% FCS and allowed to recover at 37 C, 5% CO₂ for 1-2 hours. Typical cell yields ranged from 5×10³ to 7×10⁴ stem cells per patient. Frozen solid tumors were homogenized in lysis buffer using a pestle. After centrifugation at 20,000×g, tissue homogenates were ready to use as enzyme sources. The samples were analyzed using the protocol as described in example 2. Significant individual variation in absolute levels of activity was observed (FIG. 4B). These results illustrate the usefulness of the analysis, as the activity status of PI3K/Akt pathway activation in cancer stem cells has not been assessed before due to the low cell numbers routinely obtained from patients. These data also show the feasibility of obtaining absolute units for the activity of protein kinases in small amounts of tissue material, thus demonstrating the suitability of the method for the analysis of biopsies in a clinical setting. Identification of aberrant enzymatic activity is directly useful for selecting therapies that are effective for targeting the pathway in which the enzyme operates.

### Example 8

The enzymatic activity of the lipid kinase phosphoinositide 3-kinase is measured in the following manner.

A sample from a cell lysate or purified enzyme sample having phosphoinositide 3-kinase (e.g., 0.1 to 1000 ng enzyme) is mixed with 1 to 100 mM phosphotidylinositide-4,5-bisphosphate in the presence of 0.1 to 1 mM ATP. The reaction is allowed to occur for 1 to 1000 minutes in order to produce phosphotidylinositide-3,4,5-trisphosphate in quantities sufficient enough to be detected using mass spectrometry. The reaction is stopped with the addition of a CHCl₃:CH₃OH:H₂O (1:1:0.3) solution containing 1 pmol/μL internal standard (mass labeled phosphotidylinositide-3,4,5-trisphosphate on the inositol ring or on at least one of the aliphatic chains). Aliquots (5 μL) from the reaction are analyzed by either ESI-TOF MS (Micromass/Waters Q-TOF instrument) or LC-ESI-MS/MS (Ultimate HPLC, Dionex, connected to a Micromass/Waters Q-TOF instrument) using a ESI interface in negative ion mode. Reaction products are analyzed by LC-MS/MS without further treatment or using a prior clean-up step by strong anion exchange over a polyCAT A resin (PolyLC, USA). The analysis is performed by monitoring the parent-daughter ion transition of m/z 1049 to m/z 951 for the peptide product and 1055 to 957 for the internal standard.

### Example 9

A multiplexed analysis of three different enzymes with four different substrates was performed in the following manner.

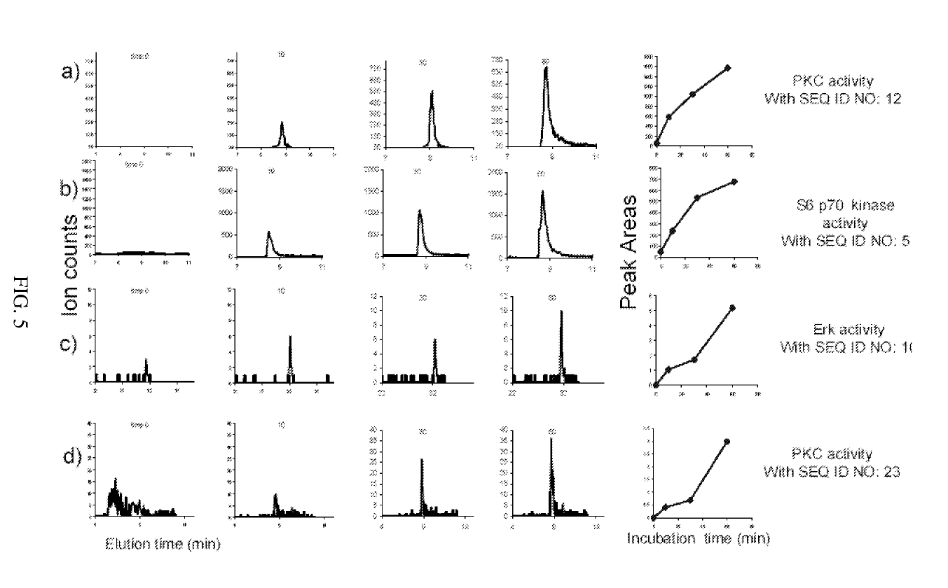

A sample of 20 ng each of recombinant PKC, recombinant S6 p70 kinase, and recombinant Erk, all purchased from Upstate (Dundee, United Kingdom), was treated with 100 μM each of SEQ ID NO: 12, SEQ ID NO: 5. SEQ ID NO: 10, and SEQ ID NO: 23 in the presence of 100 μM ATP, 5 mM magnesium chloride, 0.1 mM EDTA, 5 mM β-glycerol phosphate, 0.1 mM sodium orthovanadate, and 0.1 mM DTT in a total reaction volume of 50 μL. At time points 0, 10, 30, and 60 minutes, the reaction was stopped with the addition of 50 μL of a 1% solution of trifluoroacetic acid, and 5 μL of this mixture was analyzed by nanoflow LC-MS, in an Ultimate HPLC (Dionex/LC Packings) connected via an ESI interface to a Q-T of instrument (Waters/Micromass). The extracted mass chromatogram of each enzymatic product is shown in FIG. 5, where a) corresponds to phosphorylation of SEQ ID NO: 12 by PKC; b) corresponds to phosphylation of SEQ ID NO: 5 by S6 p70 kinase; c) corresponds to phosphorylation of SEQ ID NO: 10 by Erk; and d) corresponds to phosphorylation of SEQ ID NO: 23 by PKC. These curves were integrated and the areas under the main peak calculated using MassLynx 4.0 (Waters/Micromass) and plotted against incubation time to create the plots of the right-most column in FIG. 5.

1. (canceled)

2. A quantitative method of measuring the activity of an enzyme in a sample that contains a plurality of biologically active enzymes, the method comprising:
a) incubating the sample with a substrate composition to start an enzymatic reaction, wherein the substrate composition comprises a first substrate that is specific for a first enzyme that is known or suspected of being in the sample, and wherein the incubating is under conditions effective to permit a first reaction between the first enzyme and the first substrate to produce a first product;
b) combining an aliquot from the enzymatic reaction with a measured quantity of a first standard of known molecular weight to form a first mixture for analysis; and
c) analyzing the first mixture by liquid chromatography-mass spectrometry (LC-MS) to determine the quantity of the first product that is present in the first mixture, wherein the quantity of the first product provides a quantitative measurement of the activity of the first enzyme in the sample.

3. The method of claim 2, wherein the enzyme is a kinase and the conditions comprise including adenosine triphosphate (ATP) in the first reaction.

4. The method of claim 2, wherein the enzyme is a protein kinase.

5. (canceled)

6. The method of claim 2 wherein, in the analyzing step, the quantity of the first product is calculated by comparing mass spectrometric measurements of the first product and the first standard in the first mixture.

7. (canceled)

8. (canceled)

9. The method of claim 2, wherein the sample comprises a cell lysate that comprises enzymes from a cell.

10. The method of claim 2, wherein the analyzing of the first mixture by mass spectrometry comprises:
performing a first mass spectrometry analysis to isolate a fraction of the first mixture that contains the first product and the standard;
fragmenting the first product and the first standard in the fraction; and
performing a second mass spectrometry analysis after the fragmenting to quantitatively measure at least one fragment from the first product and the first standard, wherein the fragment measurements indicate the quantities of the first product and the first standard in the first mixture.

11. (canceled)

12. (canceled)

13. (canceled)

14. The method of claim 2, wherein the enzyme participates in a cellular signaling pathway, and the pathway is selected from the group consisting of P13K/AKT pathways; Ras/Raf/MEK/Erk pathways; MAP kinase pathways; JAK/STAT pathways; mTOR/TSC pathways; heterotrimeric G protein pathways; PKA pathways; PLC/PKC pathways; NK-kappaB pathways; cell cycle pathways (cell cycle kinases); TGF-beta pathways; TLR pathways; Notch pathways; Wnt pathways; Nutrient signaling pathways (AMPK signaling); cell-cell and cell-substratum adhesion pathways (such as cadherin, integrins); stress signaling pathways (high/low salt, heat, radiation); cytokine signaling pathways; antigen receptor signaling pathways; and co-stimulatory immune signaling pathways.

15. (canceled)

16. (canceled)

17. (canceled)

18. (canceled)

19. The method of claim 2 wherein the first substrate comprises a first peptide.

20. (canceled)

21. (canceled)

22. The method of claim 2, wherein the first standard is identical to the first product, with the proviso that the mass of the first standard differs from the mass of the first product due to incorporation of at least one isotopic label.

23. (canceled)

24. The method of claim 2, wherein the first substrate consists essentially of the amino acid residues of the first peptide.

25. The method of claim 2, wherein the sample comprises a lysate of cells from a human or animal subject.

26. The method of claim 25 wherein the sample comprises a lysate from 100 or fewer cells.

27. (canceled)

28. (canceled)

29. The method of claim 25, wherein the human or animal subject is suspected of having a disease characterized by changes in the activity of an enzyme involved in a cellular process, and wherein the enzyme involved in the cellular process is the first enzyme.

30. The method of claim 2, further comprising quantitatively detecting the activity of a second enzyme,
wherein the incubating further comprises simultaneously incubating the sample with a second substrate that is specific for a second enzyme that is known or suspected of being in the sample and that differs from the first enzyme, wherein the second enzyme modifies the second substrate in a second reaction under said conditions to form a second product; and
wherein the analyzing further comprises measuring, by mass spectrometry, the quantity of the second product produced during the incubating step, wherein the quantity of the second product provides a quantitative measurement of the activity of the second enzyme in the sample.

31. The method of claim 30, comprising mixing an aliquot from the enzymatic reaction with a measured quantity of a second standard of known molecular weight to form a second mixture for analysis.

32. The method of claim 31, wherein the first and second standards are combined with the same aliquot, whereby the first and second mixtures are the same, to permit simultaneous mass spectrometric analysis of the first and second products.

33. The method of claim 31, wherein the analyzing of the second mixture comprises comparing mass spectrometric measurements of the second product and the second standard that are present in the second mixture, to calculate the quantity of the second product that is present in the second mixture,
wherein the quantity of the second product in the second mixture provides a quantitative measurement of the activity of the second enzyme in the sample.

34. The method of claim 30, wherein the second enzyme is a protein kinase.

35. A method of screening compounds to identify a drug candidate comprising:
measuring the activity of at least one enzyme according to the method of claim 2, in the presence and absence of at least one test compound; and
comparing the activity of the at least one enzyme in the presence and absence of the at least one test compound, wherein the method identifies an inhibitor or agonist drug candidate from reduced or increased activity, respectively, of the at least one enzyme in the presence of the at least one compound.

36. A method of screening compounds to identify a drug candidate comprising
simultaneously measuring the activity of two or more enzymes according to the method of claim 2, in the presence and absence of at least one test compound and
comparing the activity of the at least two enzymes in the presence and absence of the at least one test compound, wherein the method identifies an inhibitor or agonist drug candidate from reduced or increased activity, respectively, of the at least two enzymes in the presence of the at least one test compound.

37. (canceled)

38. (canceled)

39. (canceled)

40. (canceled)

41. A method for screening an organism for a disease, disorder, or abnormality characterized by aberrant enzymatic activity, comprising:
(a) quantitatively measuring the activity of the first enzyme from a cell lysate from at least one cell of the organism, according to the method of claim 2; and
(b) comparing the measurement to a reference measurement of the activity of the first enzyme, wherein the presence or absence of the abnormality is identified from the comparison.

42. A method of characterizing a disease, disorder, or abnormality comprising:
quantitatively measuring the activity of at least one enzyme from a sample according to the method of claim 2, wherein the sample comprises at least one diseased cell isolated from a mammalian subject, or comprises a lysate of the at least one cell;
comparing the measurement(s) to a reference measurement of the activity of the at least one enzyme; and
characterizing the disease or disorder by identifying an enzyme with elevated activity in the at least one cell known or suspected of being diseased compared to activity of the enzyme in non-diseased cells of the same type as the diseased cell.

43. The method of claim 42, wherein the disease is a neoplastic disease.

44. (canceled)

45. (canceled)

46. (canceled)

47. The method of claim 42, wherein the cell or cell lysate is obtained from cells from a medical biopsy obtained from a human and snap frozen to preserve enzymatic activity.

48. The method of claim 41, wherein the reference measurement (a) is obtained from cells obtained from the same organism at a different time or from a different location in the organism, (b) is obtained from cells of the same cell type, from a different organism of the same species, or (c) is a statistical measurement calculated from measurements of samples of cells of the same cell type, from multiple organisms of the same species.

49. The method of claim 42 wherein the reference measurement (a) is obtained from cells of the same cell type, from a different organism of the same species, (b) is obtained from cells obtained from the same organism at a different time or from a different location in the organism, or (c) is a statistical measurement calculated from measurements of samples of cells of the same cell type, from multiple organisms of the same species.

50. (canceled)

51. (canceled)

52. The method of claim 41 wherein the disease, disorder, or abnormality is cancer.

53. (canceled)

54. (canceled)

55. A quantitative method of detecting the activity of a signaling pathway in a sample having a plurality of biologically active enzymes comprising
a) incubating the sample with a substrate composition to start an enzymatic reaction, wherein the substrate composition comprises a first substrate that is specific for the signaling pathway, and wherein the incubating is under conditions effective to permit a first reaction between at least one enzyme of the signaling pathway and the first substrate to produce a first product;
b) combining an aliquot from the reaction with a measured quantity of a first standard of known molecular weight to form a first mixture for analysis; and
c) analyzing the first mixture by mass spectrometry to determine the quantity of the first product that is present in the first mixture,
wherein the quantity of the first product provides a quantitative measurement of the activity of the signaling pathway in the sample.

56. A kit comprising
(a) a plurality of substrate containers, wherein each substrate container contains at least one enzymatic peptide substrate that an enzyme modifies to form a product; and
(b) a plurality of standard containers, wherein each standard container contains at least one mass labeled standard of a known concentration, and wherein each mass labeled standard is identical to one of the products, with the proviso that the product and the standard have different molecular weights due to isotopic labeling of the standard or the product.

57. (canceled)

58. (canceled)

59. (canceled)

60. A composition comprising a mixture of two or more peptide standards of known molecular weight and concentration, wherein each of the standards comprises a chemical structure identical to an enzyme product and a molecular weight different than the enzyme product due to incorporation of at least one isotopic label in the standard.

61. (canceled)

62. (canceled)

63. (canceled)
